(19) United States
(12) Patent Application Publication
(10) Pub. No.: US 2026/0115437 A1
(43) Pub. Date: Apr. 30, 2026

(54) INTRANASAL KNOCKOUT BOLUS

(71) Applicant: Rocket Science Health Corp., Victoria (CA)

(72) Inventors: Kenneth IRVING, Victoria (CA); Alec Lillis, Victoria (CA); Timothy Rees, Victoria (CA); Hilary Egglestone, Victoria (CA); Manu Sharma, JR., Victoria (CA)

(21) Appl. No.: 19/406,289

(22) Filed: Dec. 2, 2025

Related U.S. Application Data
(63) Continuation of application No. PCT/CA2025/050023, filed on Jan. 9, 2025.
(60) Provisional application No. 63/619,560, filed on Jan. 10, 2024.

(51) Int. Cl. A61M 31/00; A61M 3/02

(52) U.S. Cl. CPC A61M 31/00; A61M 3/0279; A61M 2206/11; A61M 2210/0618

(57) ABSTRACT

The present disclosure describes intranasal knockout bolus formulations, methods, and devices. Olfactory delivery of a therapeutic agent via an intranasal bolus of the therapeutic agent may be reduced or terminated by precision delivery of a knockout bolus of a purging formulation. The knockout bolus may be delivered at a velocity sufficient to physically displace the therapeutic agent from an olfactory cleft, may dilute the intranasal bolus of a therapeutic formulation, or may cause an osmotic pressure sufficient to reverse a direction of a drug flow through a tissue of the olfactory cleft. An ability to modulate or stop delivery of the therapeutic agent via the olfactory cleft allows for more precision dosing and more control of dose timing in response to biometric monitoring.

### CROSS REFERENCE

This application is a continuation application of International Application No. PCT/CA2025/050023, filed Jan. 9, 2025, which claims the benefit to U.S. Provisional Application No. 63/619,560, filed Jan. 10, 2024, the contents of which are incorporated herein in their entireties by reference.

### BACKGROUND

Intranasal dose regulation is consistently a significant concern when contemplating intranasal delivery of a therapeutic agent. Precision dosing is often critical, and delivery of a therapeutic formulation to the olfactory cleft produces effects which vary over time, based on the relevant pharmacokinetics and pharmacodynamics. Intranasal therapeutic formulations may remain longer than intended. Even after the desired pharma-dynamic effect has been achieved, formulations in the nasal cavity may continue to transfer drugs to the patient's tissue. Substances remaining in the olfactory cleft (or neighboring sites) may continue to transfer to the central nervous system. This may increase the risk of overdosing, and other undesirable effects.

There is a need for formulations, methods, and devices for purging of the olfactory cleft.

### SUMMARY

It is appreciated by the inventors that there are situations in which reduction or termination of the olfactory delivery of a therapeutic agent may be desirable. Methods are described herein wherein a drug deposited within the nasal cavity is expelled using a secondary liquid injected into an intranasal region at a given velocity. Displacement of the drug may be driven by the pressure exerted by the incoming liquid injection and may be further enhanced by the specific properties of the liquid, such as density, viscosity, and surfactant presence. In some embodiments, dose-knockout methods for intranasal drug delivery and drug expulsion methods for the nasal cavity are described herein.

There are many possible advantages of drug removal. As a first example, it provides a means to regulate the dosage delivered to the patient by removing the drug deposit once a sufficient dose has been absorbed. Natural clearance processes may be inadequate to restrict residence time for some high-concentration doses of medicine. As a second example, it offers a method to clear highly viscous or adhesive drug carrier remnants from the nasal cavity after they have been depleted of their drug content. The method may also slow the drug transfer rates based on the principles of dilution, concentration gradients, and possibly osmosis. If the knockout formulation is the same as the drug-bearing formulation minus the active ingredient, its arrival will dilute the bolus in place, lowering the total concentration of available drug. This, in turn, may reduce or even reverse the flow of drug if osmotic pressures are sufficient.

In some cases, the knockout bolus may be delivered at a time selected in response to a monitoring of the effects of the therapeutic agent delivered via the olfactory cleft. The site of action for the therapeutic agent may be very different from the tissue of absorption, so various biometric monitoring techniques may be appropriate.

Drug expulsion methods described herein may help to address the challenge of intranasal dose regulation. While intranasal drug deposition formulations are sometimes designed with adhesion as an objective, drug expulsion formulations may be tailored for swift clearance of the region.

Additionally, in some multi-drug treatments, it may be essential to purge the first medicine before subsequent formulations may be delivered, with timing as a possible factor. Similarly, for some therapeutic agents, there may be advantage to purging the olfactory cleft prior to delivery of a first therapeutic formulation.

In one aspect, provided herein is a method of delivery of a therapeutic formulation via an olfactory cleft of a patient, the method comprising: (a) delivering the therapeutic formulation to the olfactory cleft; (b) waiting for a duration of time; and (c) delivering a purging formulation to the olfactory cleft, thereby purging the therapeutic formulation from the olfactory cleft.

In another aspect, provided herein is a method of purging an olfactory cleft of a patient, the method comprising: (a) providing a purging formulation configured to purge the olfactory cleft; and (b) delivering the purging formulation to the olfactory cleft, thereby purging the olfactory cleft.

In another aspect, provided herein is a method of purging left and right olfactory clefts of a patient, the method comprising: providing a left purging formulation configured to purge the left olfactory cleft; providing a right purging formulation configured to purge the right olfactory cleft; delivering the left purging formulation to the left olfactory cleft, thereby purging the left olfactory cleft; and delivering the right purging formulation to the right olfactory cleft, thereby purging the right olfactory cleft.

In another aspect, provided herein is a method of controlling delivery of a therapeutic formulation via an olfactory cleft of a patient, the method comprising: (a) delivering a first volume of a therapeutic formulation to the olfactory cleft; (b) monitoring a response to the therapeutic formulation; and (c) delivering one or more additional volumes of the therapeutic formulation to the olfactory cleft based on the monitored response to the therapeutic formulation.

In another aspect, provided herein is a device of purging an olfactory cleft of a patient, the device comprising: a housing comprising an insertable portion comprising a distal end, and a proximal end; and a subject-engaging portion which engages a columella region of the subject to seat the distal end of the insertable portion within an ejection zone of a nasal channel of the subject; wherein the device is configured to deliver a purging formulation to the olfactory cleft of the patient, thereby purging the olfactory cleft.

In yet another aspect, disclosed herein is a device for controlled delivery of a therapeutic formulation via an olfactory cleft of a patient, the device comprising: a first chamber configured to hold the therapeutic formulation; a second chamber configured to hold a purging formulation for purging the therapeutic formulation from the olfactory cleft; and a delivery mechanism configured to sequentially deliver the therapeutic formulation and the purging formulation to the olfactory cleft.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description and accompanying drawings, that set forth illustrative embodiments in which the principles of the present disclosure are utilized.

FIG. 1A depicts a device having a dispensing mechanism configured to dispense a therapeutic formulation and purging formulation through a dispensing tip for sequential delivery to an olfactory cleft of a patient.

FIG. 1B depicts the device shown in FIG. 1A once the therapeutic formulation is delivered to, and remains in, the olfactory cleft.

FIG. 1C depicts the device shown in FIG. 1B once the purging formulation displaces the therapeutic formulation from the olfactory cleft.

FIG. 1D depicts the device shown in FIG. 1C once the purging formulation is eliminated from the olfactory cleft, leaving an empty olfactory cleft.

FIG. 2A depicts a front, base and perspective view of an exemplary embodiment of a subject's nose and the columella region.

FIG. 2B depicts a side view of an exemplary embodiment of a representative subject's nasal cavity, including internal anatomical features.

FIG. 2C depicts an exemplary embodiment of an Exemplary Device along a side view of the sagittal plane of delivery to the target region in the subject, according to some embodiments.

FIG. 2D depicts an exemplary embodiment of an Exemplary Device along a side view of the sagittal plane aiming from the respiratory region in the subject, according to some embodiments.

FIG. 2E depicts a side view of an ejection zone, according to some embodiments.

FIG. 2F depicts a side view of an ejection zone with respect to other nasal cavity anatomy, according to some embodiments.

FIGS. 3A-3C are images of a transparent model nasal cavity depicting knocking out of a primary dose (dark formulation) by a secondary dose (white formulation) according to some embodiments. Both doses had a viscosity of 1 cP. (A) Primary dose depositing at the olfactory. (B) Secondary dose interacting with the primary dose. (C) Primary dose knocked out by the secondary dose.

FIGS. 4A-4C are images of a transparent model nasal cavity depicting knocking out of a primary dose (dark formulation) by a secondary dose (white formulation) according to some embodiments. Both doses had a viscosity of 20 cP. (A) Primary dose depositing at the olfactory. (B) Secondary dose interacting with the primary dose. (C) Primary dose knocked out by the secondary dose.

FIGS. 5A-5C are images of a transparent model nasal cavity depicting knocking out of a primary dose (dark formulation) by a secondary dose (white formulation) according to some embodiments. The primary dose and the secondary dose had a viscosity of 20 cP and 1 cP, respectively. (A) Primary dose depositing at the olfactory. (B) Secondary dose interacting with the primary dose. (C) Primary dose knocked out by the secondary dose.

FIGS. 6A-6C are images of a transparent model nasal cavity depicting knocking out of a primary dose (dark formulation) by a secondary dose (white formulation) according to some embodiments. The primary dose and the secondary dose had a viscosity of 1 cP and 20 cP, respectively. (A) Primary dose depositing at the olfactory. (B) Secondary dose interacting with the primary dose. (C) Primary dose knocked out by the secondary dose.

FIG. 7 depicts a device for use with the method disclosed herein in certain embodiments.

### DETAILED DESCRIPTION

Some portions of the nasal anatomy may be rinsed relatively easily via passage of a rinse fluid through the nasal passage. For example, simple nasal irrigation devices such as neti pots have long been used to rinse parts of the intranasal cavity by flowing water into one nostril and out the other nostril. However, certain intranasal regions, such as the olfactory cleft, are not as easily rinsed, due to the complex intranasal geometry. The olfactory cleft is a unique part of the intranasal anatomy that is effectively a dead-end. Accordingly, precisely directed olfactory purging may be much more efficient than general flow-through intranasal rinsing. Additionally, depending on the characteristics of the particular drug-laden bolus to be purged, the knockout fluid may require certain properties (such as a certain viscosity, density, or surface tension). In some situations, the knockout liquid may also require surfactants to lessen the surface tension of the drug-laden bolus. In some embodiments, by introducing fluid at a specific velocity into a confined space containing a therapeutic formulation, the increased pressure from the incoming fluid propels the therapeutic formulation to flow towards the exit, where the pressure is comparatively lower.

Traditional intranasal rinsing devices may lack the ability to efficiently purge the olfactory cleft. For example, rinse fluids delivered via some devices may lack the velocity and/or specific direction to access the olfactory cleft at all and may fail to successfully purge a bolus positioned within or around the olfactory cleft.

Intranasal expulsion of a therapeutic formulation after delivery provides the ability to set a time limit on the contact between a drug and an absorption site. This can allow delivery of formulations with higher concentrations of active ingredients, with limited side effects, providing a dosage control in some ways similar to drug patches.

### I. Methods

Accordingly, in one aspect, provided herein is a method of delivery of a therapeutic formulation via an olfactory cleft of a patient, the method comprising: (a) delivering the therapeutic formulation to the olfactory cleft; and (b) delivering a purging formulation to the olfactory cleft, thereby purging the therapeutic formulation from the olfactory cleft. In one aspect, provided herein is a method of delivery of a therapeutic formulation via an olfactory cleft of a patient, the method comprising: (a) delivering the therapeutic formulation to the olfactory cleft; (b) waiting for a duration of time; and (c) delivering a purging formulation to the olfactory cleft, thereby purging the therapeutic formulation from the olfactory cleft. In one aspect, disclosed herein is a method of delivery of a therapeutic formulation. The delivery can be via an olfactory cleft of a patient. The method can comprise delivering the therapeutic formulation to the olfactory cleft. The method can comprise waiting for a duration of time. The method can comprise delivering a purging formulation to the olfactory cleft, thereby purging the therapeutic formulation from the olfactory cleft.

In another aspect, provided herein is a method of purging an olfactory cleft of a patient, the method comprising: (a) providing a purging formulation configured to purge the olfactory cleft; and (b) delivering the purging formulation to the olfactory cleft, thereby purging the olfactory cleft. In one aspect, provided herein is a method of purging an olfactory cleft of a patient. The method can comprise providing a purging formulation. The purging formulation can be configured to purge the olfactory cleft. The method can comprise delivering the purging formulation to the olfactory cleft, thereby purging the olfactory cleft.

In some embodiments, the purging formulation purges a therapeutic formulation from the olfactory cleft.

As used herein, the term “purging”, “purge” or the like means displacing at least a portion of a substance from an original location. The original location can, for example, be the olfactory cleft.

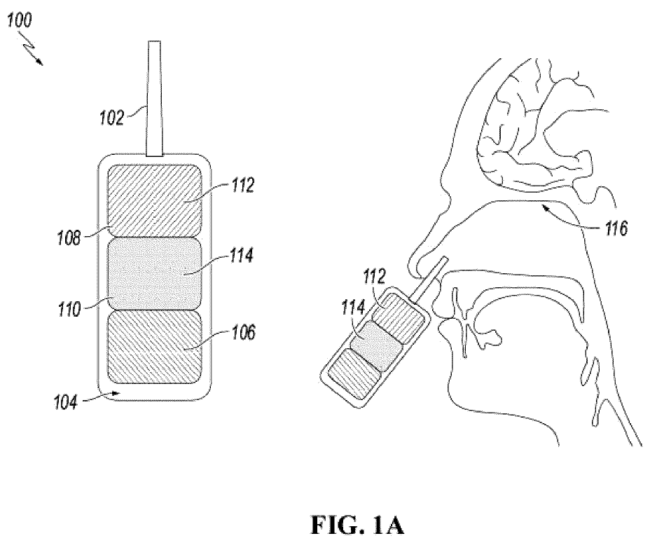

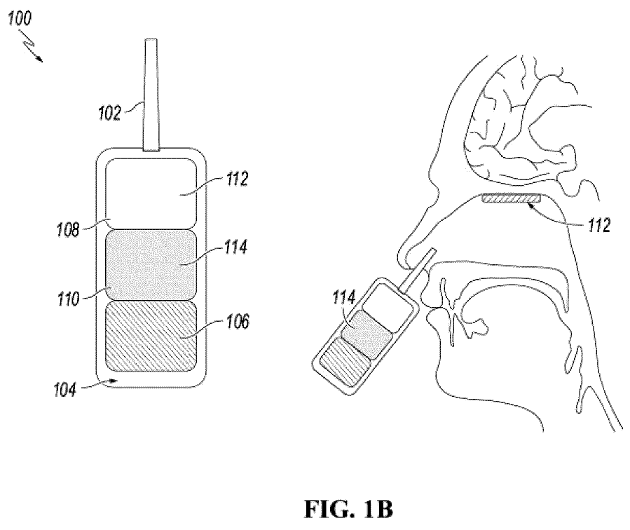

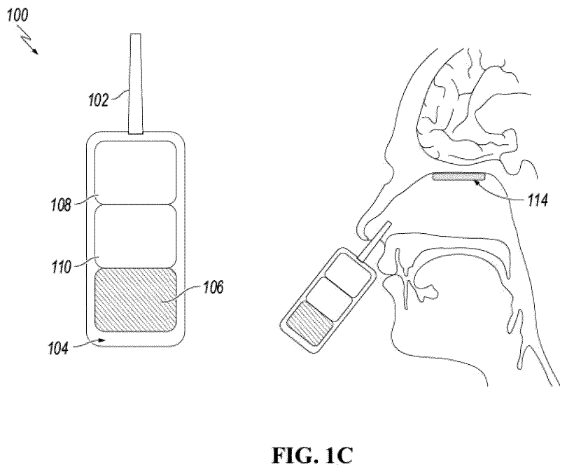

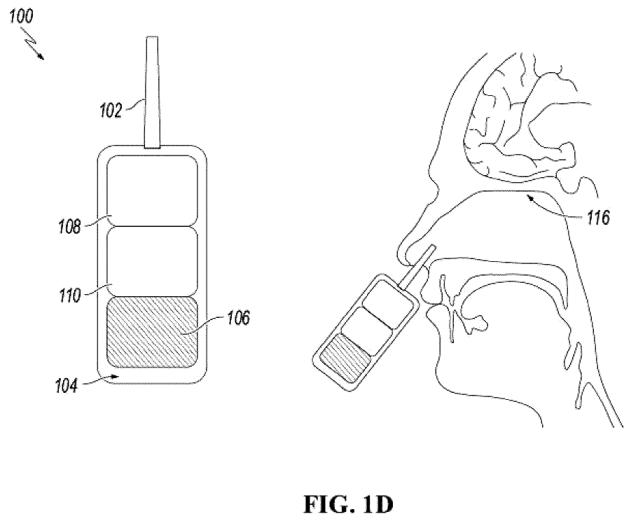

Referring to FIGS. 1A-1D, which depict a device having a dispensing mechanism configured to dispense a therapeutic formulation and purging formulation through a dispensing tip for sequential delivery to an olfactory cleft of a patient.

FIG. 1A depicts an example device 100 according to some embodiments. The device 100 comprises a dispensing tip 102, an upper reservoir 108, a lower reservoir 110, and a device enclosure 104. A therapeutic formulation 112 is stored in the upper reservoir 108, and a purging formulation 114 is stored in the lower reservoir 110. In some embodiments, the device 100 further comprises a dispensing mechanism 106 for sequential delivery of the therapeutic formulation 112 and the purging formulation 114. In some embodiments, delivery of the therapeutic formulation 112 results in the therapeutic formulation 112 at least partially filling the olfactory cleft 116 (FIG. 1A). In some embodiments, after a duration of time, the purging formulation is dispensed from the device 100, and displaces the therapeutic formulation 112 from the olfactory cleft 116 (FIG. 1C and FIG. 1D).

The therapeutic formulation and/or the purging formulation can be delivered as a liquid jet. The liquid jet can be a laminar flow. The therapeutic formulation and/or the purging formulation can precisely target the olfactory cleft of the patient. Targeted delivery of a composition to the olfactory cleft is described in WO2024224372, the content of which is incorporated herein by reference. For example, the therapeutic formulation and/or the purging formulation can be delivered from a position sufficiently deep within the nasal cavity to allow for precise targeting of the olfactory cleft or another intranasal region. Delivering the therapeutic formulation can comprise ejecting the therapeutic formulation from a delivery device. Delivering the purging formulation can comprise ejecting the purging formulation from a delivery device. The therapeutic formulation and the purging formulation can be ejected from different delivery devices. The therapeutic formulation and the purging formulation can be ejected from the same delivery device. The therapeutic formulation and/or the purging formulation can be ejected from an ejection zone in a nasal cavity of the patient.

In some embodiments, the purging formulation is ejected from a delivery device as a liquid jet. In some embodiments, the purging formulation is ejected from a delivery device as a laminar flow. In some embodiments, the purging formulation is ejected from a delivery device as a flow having a Reynold's number of 2300 or less. In some embodiments, the purging formulation is ejected from a delivery device as a flow having a Reynold's number of 2200 or less. In some embodiments, the purging formulation is ejected from a delivery device as a flow having a Reynold's number of 2100 or less. In some embodiments, the purging formulation is ejected from a delivery device as a flow having a Reynold's number of 2000 or less. In some embodiments, the purging formulation is ejected from a delivery device as a flow having a Reynold's number of 1900 or less. In some embodiments, the purging formulation is ejected from a delivery device as a flow having a Reynold's number of 1800 or less. In some embodiments, the purging formulation is ejected from a delivery device as a flow having a Reynold's number of 1700 or less. In some embodiments, the purging formulation is ejected from a delivery device as a flow having a Reynold's number of 1600 or less. In some embodiments, the purging formulation is ejected from a delivery device as a flow having a Reynold's number of 1500 or less. In some embodiments, the purging formulation is ejected from a delivery device as a flow having a Reynold's number of 1400 or less. In some embodiments, the purging formulation is ejected from a delivery device as a flow having a Reynold's number of 1300 or less. In some embodiments, the purging formulation is ejected from a delivery device as a flow having a Reynold's number of 1200 or less. In some embodiments, the purging formulation is ejected from a delivery device as a flow having a Reynold's number of 1100 or less. In some embodiments, the purging formulation is ejected from a delivery device as a flow having a Reynold's number of 1000 or less. In some embodiments, the purging formulation is ejected from a delivery device as a flow having a Reynold's number of 900 or less. In some embodiments, the purging formulation is ejected from a delivery device as a flow having a Reynold's number of 800 or less. In some embodiments, the purging formulation is ejected from a delivery device as a flow having a Reynold's number of 700 or less. In some embodiments, the purging formulation is ejected from a delivery device as a flow having a Reynold's number of 600 or less. In some embodiments, the purging formulation is ejected from a delivery device as a flow having a Reynold's number of 500 or less. In some embodiments, the purging formulation is ejected from a delivery device as a flow having a Reynold's number of 400 or less. In some embodiments, the purging formulation is ejected from a delivery device as a flow having a Reynold's number of 300 or less.

In some embodiments, the purging formulation precisely targets the olfactory cleft. In some embodiments, the purging formulation is delivered from a position sufficiently deep within the nasal cavity to allow for precise targeting of the olfactory cleft or another intranasal region.

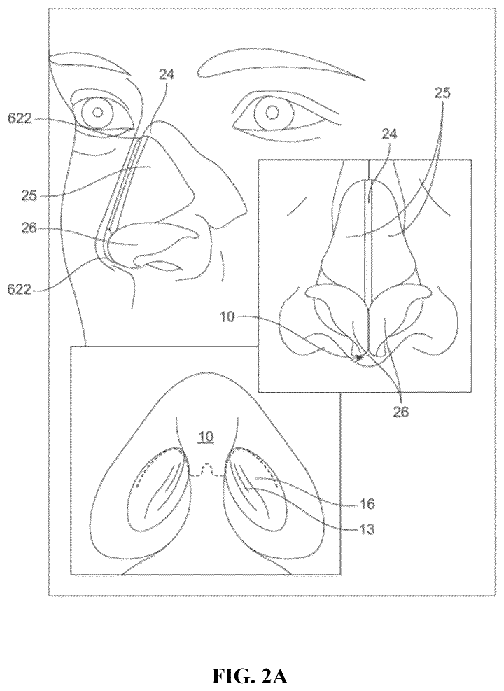

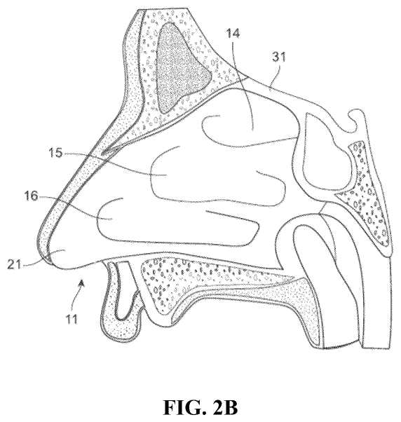

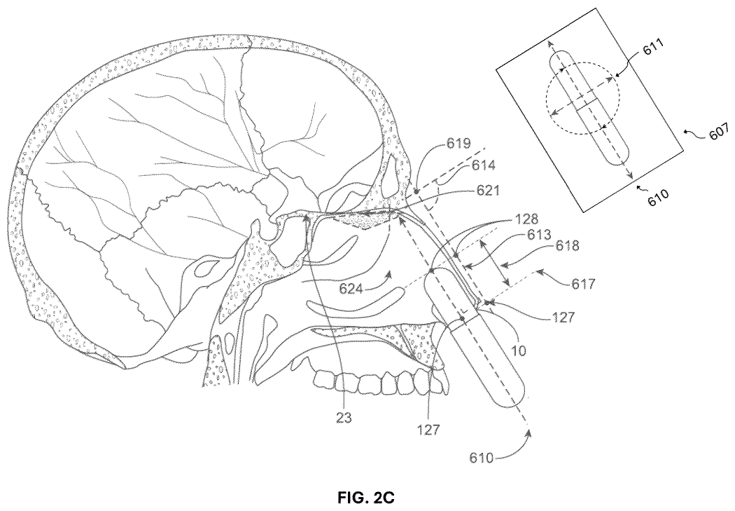

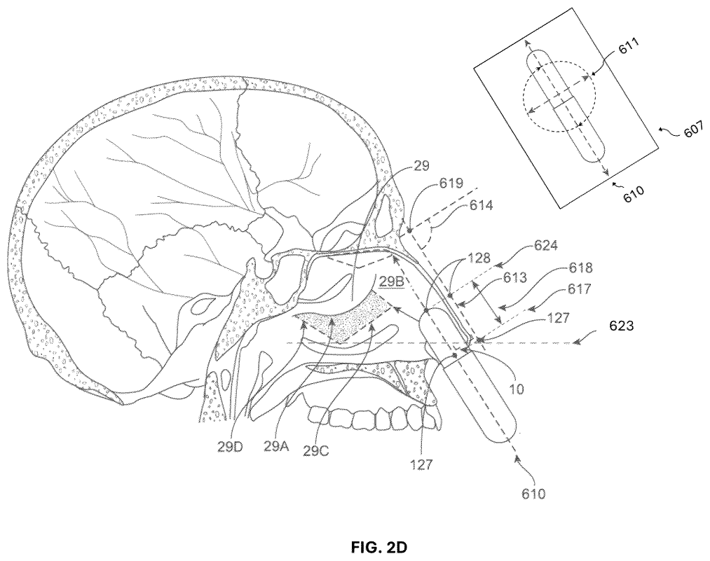

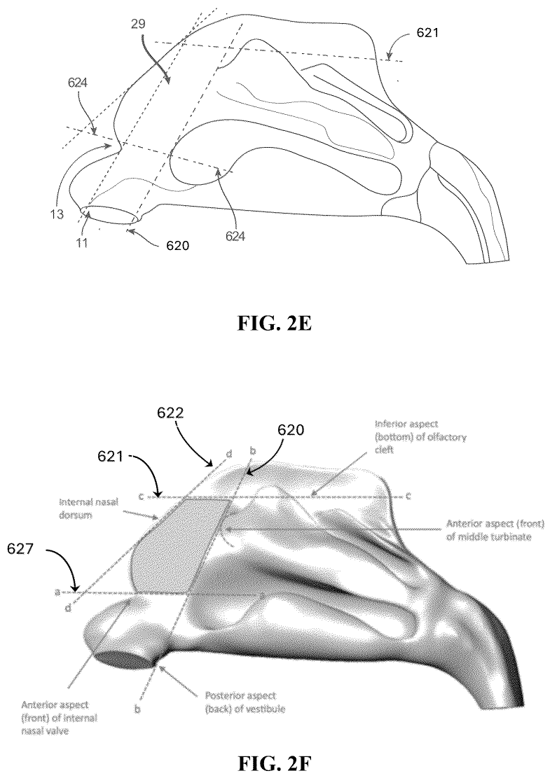

Referring to FIGS. 2A-2F. In some embodiments, the ejection zone 29 is: 0 mm to 30 mm superior to a horizontal line 627 that intersects the anterior aspect of the internal nasal valve 13, and 0 mm to 20 mm anterior to an inclined line 620 that intersects the anterior aspect of the middle turbinate 15 and the posterior aspect of the vestibule 21.

In some embodiments, the ejection zone 29 is further 0 mm to 40 mm inferior to a horizontal line 621 that is parallel to the inferior aspect of the olfactory cleft 23, 0 mm to 20 mm posterior to the internal nasal dorsum 622, 10 mm to 50 mm superior to a horizontal line 623 that intersects the inferior aspect of the columella 10, 0 mm to 30 mm superior to a horizontal line 624 that intersects the superior aspect of the inferior turbinate 16, 0 mm to 3 mm from the septum 24, or any combination thereof.

In some embodiments, the ejection zone 29 is a trapezium or irregular quadrilateral comprising (i) an inferior side 29A being a 10-25 mm line extending posteriorly and horizontally from the anterior aspect of the internal nasal valve 13, (ii) an anterior side 29B being a 10-35 mm line extending superiorly and parallel to the internal nasal dorsum 622 from the anterior aspect of the internal nasal valve 13, (iii) a superior side 29C being a 10-25 mm line extending posteriorly and horizontally from a point on the internal nasal dorsum 622 that is 0-10 mm inferior to the inferior aspect of the olfactory cleft 23, and (iv) a posterior line 29D being a 10-35 mm line extending vertically along a plane that intersects the anterior aspect of the middle turbinate 15.

In some embodiments, the purging formulation is ejected from an ejection zone in a nasal cavity of the subject, wherein the ejection zone 29 is: 0 mm to 30 mm superior to a horizontal line 627 that intersects the anterior aspect of the internal nasal valve 13, and 0 mm to 20 mm anterior to an inclined line 620 that intersects the anterior aspect of the middle turbinate 15 and the posterior aspect of the vestibule 21, and one or more of the following: 0 mm to 40 mm inferior to a horizontal line 621 that is parallel to the inferior aspect of the olfactory cleft 23, 0 mm to 20 mm posterior to the internal nasal dorsum 622, 10 mm to 50 mm superior to a horizontal line 623 that intersects the inferior aspect of the columella 10, 0 mm to 30 mm superior to a horizontal line 624 that intersects the superior aspect of the inferior turbinate 16, 0 mm to 3 mm from the septum 24 or any combination thereof.

In some embodiments, the ejection zone is superior to a horizontal line that intersects the anterior aspect of the internal nasal valve by about 1 mm to about 30 mm. In some embodiments, the ejection zone is superior to a horizontal line that intersects the anterior aspect of the internal nasal valve by about 1 mm to about 2 mm, about 1 mm to about 4 mm, about 1 mm to about 6 mm, about 1 mm to about 8 mm, about 1 mm to about 10 mm, about 1 mm to about 14 mm, about 1 mm to about 18 mm, about 1 mm to about 22 mm, about 1 mm to about 26 mm, about 1 mm to about 30 mm, about 2 mm to about 4 mm, about 2 mm to about 6 mm, about 2 mm to about 8 mm, about 2 mm to about 10 mm, about 2 mm to about 14 mm, about 2 mm to about 18 mm, about 2 mm to about 22 mm, about 2 mm to about 26 mm, about 2 mm to about 30 mm, about 4 mm to about 6 mm, about 4 mm to about 8 mm, about 4 mm to about 10 mm, about 4 mm to about 14 mm, about 4 mm to about 18 mm, about 4 mm to about 22 mm, about 4 mm to about 26 mm, about 4 mm to about 30 mm, about 6 mm to about 8 mm, about 6 mm to about 10 mm, about 6 mm to about 14 mm, about 6 mm to about 18 mm, about 6 mm to about 22 mm, about 6 mm to about 26 mm, about 6 mm to about 30 mm, about 8 mm to about 10 mm, about 8 mm to about 14 mm, about 8 mm to about 18 mm, about 8 mm to about 22 mm, about 8 mm to about 26 mm, about 8 mm to about 30 mm, about 10 mm to about 14 mm, about 10 mm to about 18 mm, about 10 mm to about 22 mm, about 10 mm to about 26 mm, about 10 mm to about 30 mm, about 14 mm to about 18 mm, about 14 mm to about 22 mm, about 14 mm to about 26 mm, about 14 mm to about 30 mm, about 18 mm to about 22 mm, about 18 mm to about 26 mm, about 18 mm to about 30 mm, about 22 mm to about 26 mm, about 22 mm to about 30 mm, or about 26 mm to about 30 mm, including increments therein. In some embodiments, the ejection zone is superior to a horizontal line that intersects the anterior aspect of the internal nasal valve by about 1 mm, about 2 mm, about 4 mm, about 6 mm, about 8 mm, about 10 mm, about 14 mm, about 18 mm, about 22 mm, about 26 mm, or about 30 mm. In some embodiments, the ejection zone is superior to a horizontal line that intersects the anterior aspect of the internal nasal valve by at least about 1 mm, about 2 mm, about 4 mm, about 6 mm, about 8 mm, about 10 mm, about 14 mm, about 18 mm, about 22 mm, or about 26 mm. In some embodiments, the ejection zone is superior to a horizontal line that intersects the anterior aspect of the internal nasal valve by at most about 2 mm, about 4 mm, about 6 mm, about 8 mm, about 10 mm, about 14 mm, about 18 mm, about 22 mm, about 26 mm, or about 30 mm. In some embodiments, the ejection zone is anterior to an inclined line that intersects the anterior aspect of the middle turbinate and the posterior aspect of the vestibule by about 1 mm to about 20 mm. In some embodiments, the ejection zone is anterior to an inclined line that intersects the anterior aspect of the middle turbinate and the posterior aspect of the vestibule by about 1 mm to about 2 mm, about 1 mm to about 4 mm, about 1 mm to about 6 mm, about 1 mm to about 8 mm, about 1 mm to about 10 mm, about 1 mm to about 12 mm, about 1 mm to about 14 mm, about 1 mm to about 16 mm, about 1 mm to about 18 mm, about 1 mm to about 20 mm, about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or about 18 mm to about 20 mm, including increments therein. In some embodiments, the ejection zone is anterior to an inclined line that intersects the anterior aspect of the middle turbinate and the posterior aspect of the vestibule by about 1 mm, about 2 mm, about 4 mm, about 6 mm, about 8 mm, about 10 mm, about 12 mm, about 14 mm, about 16 mm, about 18 mm, or about 20 mm. In some embodiments, the ejection zone is anterior to an inclined line that intersects the anterior aspect of the middle turbinate and the posterior aspect of the vestibule by at least about 1 mm, about 2 mm, about 4 mm, about 6 mm, about 8 mm, about 10 mm, about 12 mm, about 14 mm, about 16 mm, or about 18 mm. In some embodiments, the ejection zone is anterior to an inclined line that intersects the anterior aspect of the middle turbinate and the posterior aspect of the vestibule by at most about 2 mm, about 4 mm, about 6 mm, about 8 mm, about 10 mm, about 12 mm, about 14 mm, about 16 mm, about 18 mm, or about 20 mm. In some embodiments, the ejection zone is inferior to a horizontal line that is parallel to the inferior aspect of the olfactory cleft by about 1 mm to about 35 mm. In some embodiments, the ejection zone is inferior to a horizontal line that is parallel to the inferior aspect of the olfactory cleft by about 1 mm to about 2 mm, about 1 mm to about 3 mm, about 1 mm to about 4 mm, about 1 mm to about 5 mm, about 1 mm to about 8 mm, about 1 mm to about 12 mm, about 1 mm to about 15 mm, about 1 mm to about 18 mm, about 1 mm to about 21 mm, about 1 mm to about 25 mm, about 1 mm to about 30 mm, about 1 mm to about 35 mm, about 2 mm to about 3 mm, about 2 mm to about 4 mm, about 2 mm to about 5 mm, about 2 mm to about 8 mm, about 2 mm to about 12 mm, about 2 mm to about 15 mm, about 2 mm to about 18 mm, about 2 mm to about 21 mm, about 2 mm to about 25 mm, about 2 mm to about 30 mm, about 2 mm to about 35 mm, about 3 mm to about 4 mm, about 3 mm to about 5 mm, about 3 mm to about 8 mm, about 3 mm to about 12 mm, about 3 mm to about 15 mm, about 3 mm to about 18 mm, about 3 mm to about 21 mm, about 3 mm to about 25 mm, about 3 mm to about 30 mm, about 3 mm to about 35 mm, about 4 mm to about 5 mm, about 4 mm to about 8 mm, about 4 mm to about 12 mm, about 4 mm to about 15 mm, about 4 mm to about 18 mm, about 4 mm to about 21 mm, about 4 mm to about 25 mm, about 4 mm to about 30 mm, about 4 mm to about 35 mm, about 5 mm to about 8 mm, about 5 mm to about 12 mm, about 5 mm to about 15 mm, about 5 mm to about 18 mm, about 5 mm to about 21 mm, about 5 mm to about 25 mm, about 5 mm to about 30 mm, about 5 mm to about 35 mm, about 8 mm to about 15 mm, about 8 mm to about 18 mm, about 8 mm to about 21 mm, about 8 mm to about 25 mm, about 8 mm to about 30 mm, about 8 mm to about 35 mm, about 12 mm to about 15 mm, about 12 mm to about 18 mm, about 12 mm to about 21 mm, about 12 mm to about 25 mm, about 12 mm to about 30 mm, about 12 mm to about 35 mm, about 15 mm to about 18 mm, about 15 mm to about 21 mm, about 15 mm to about 25 mm, about 15 mm to about 30 mm, about 15 mm to about 35 mm, about 18 mm to about 21 mm, about 18 mm to about 25 mm, about 18 mm to about 30 mm, about 18 mm to about 35 mm, about 21 mm to about 25 mm, about 21 mm to about 30 mm, about 21 mm to about 35 mm, about 25 mm to about 30 mm, about 25 mm to about 35 mm, or about 30 mm to about 35 mm, including increments therein. In some embodiments, the ejection zone is inferior to a horizontal line that is parallel to the inferior aspect of the olfactory cleft by about 1 mm, about 2 mm, about 3 mm, about 4 mm, about 5 mm, about 8 mm, about 12 mm, about 15 mm, about 18 mm, about 21 mm, about 25 mm, about 30 mm, or about 35 mm. In some embodiments, the ejection zone is inferior to a horizontal line that is parallel to the inferior aspect of the olfactory cleft by at least about 1 mm, about 2 mm, about 3 mm, about 4 mm, about 5 mm, about 8 mm, about 12 mm, about 15 mm, about 18 mm, about 21 mm, about 25 mm, or about 30 mm. In some embodiments, the ejection zone is inferior to a horizontal line that is parallel to the inferior aspect of the olfactory cleft by at most about 2 mm, about 3 mm, about 4 mm, about 5 mm, about 8 mm, about 12 mm, about 15 mm, about 18 mm, about 21 mm, about 25 mm, about 30 mm, or about 35 mm. In some embodiments, the ejection zone is posterior to the internal nasal dorsum by about 1 mm to about 20 mm. In some embodiments, the ejection zone is posterior to the internal nasal dorsum by about 1 mm to about 2 mm, about 1 mm to about 3 mm, about 1 mm to about 4 mm, about 1 mm to about 6 mm, about 1 mm to about 8 mm, about 1 mm to about 10 mm, about 1 mm to about 12 mm, about 1 mm to about 14 mm, about 1 mm to about 16 mm, about 1 mm to about 18 mm, about 1 mm to about 20 mm, about 2 mm to about 3 mm, about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 3 mm to about 4 mm, about 3 mm to about 6 mm, about 3 mm to about 8 mm, about 3 mm to about 10 mm, about 3 mm to about 12 mm, about 3 mm to about 14 mm, about 3 mm to about 16 mm, about 3 mm to about 18 mm, about 3 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or about 18 mm to about 20 mm, including increments therein. In some embodiments, the ejection zone is posterior to the internal nasal dorsum by about 1 mm, about 2 mm, about 3 mm, about 4 mm, about 6 mm, about 8 mm, about 10 mm, about 12 mm, about 14 mm, about 16 mm, about 18 mm, or about 20 mm. In some embodiments, the ejection zone is posterior to the internal nasal dorsum by at least about 1 mm, about 2 mm, about 3 mm, about 4 mm, about 6 mm, about 8 mm, about 10 mm, about 12 mm, about 14 mm, about 16 mm, or about 18 mm. In some embodiments, the ejection zone is posterior to the internal nasal dorsum by at most about 2 mm, about 3 mm, about 4 mm, about 6 mm, about 8 mm, about 10 mm, about 12 mm, about 14 mm, about 16 mm, about 18 mm, or about 20 mm. In some embodiments, the ejection zone is superior to a horizontal line that intersects the inferior aspect of the columella by about 10 mm to about 50 mm. In some embodiments, the ejection zone is superior to a horizontal line that intersects the inferior aspect of the columella by about 10 mm to about 12 mm, about 10 mm to about 16 mm, about 10 mm to about 20 mm, about 10 mm to about 24 mm, about 10 mm to about 28 mm, about 10 mm to about 32 mm, about 10 mm to about 36 mm, about 10 mm to about 40 mm, about 10 mm to about 45 mm, about 10 mm to about 50 mm, about 12 mm to about 16 mm, about 12 mm to about 20 mm, about 12 mm to about 24 mm, about 12 mm to about 28 mm, about 12 mm to about 32 mm, about 12 mm to about 36 mm, about 12 mm to about 40 mm, about 12 mm to about 45 mm, about 12 mm to about 50 mm, about 16 mm to about 20 mm, about 16 mm to about 24 mm, about 16 mm to about 28 mm, about 16 mm to about 32 mm, about 16 mm to about 36 mm, about 16 mm to about 40 mm, about 16 mm to about 45 mm, about 16 mm to about 50 mm, about 20 mm to about 24 mm, about 20 mm to about 28 mm, about 20 mm to about 32 mm, about 20 mm to about 36 mm, about 20 mm to about 40 mm, about 20 mm to about 45 mm, about 20 mm to about 50 mm, about 24 mm to about 28 mm, about 24 mm to about 32 mm, about 24 mm to about 36 mm, about 24 mm to about 40 mm, about 24 mm to about 45 mm, about 24 mm to about 50 mm, about 28 mm to about 32 mm, about 28 mm to about 36 mm, about 28 mm to about 40 mm, about 28 mm to about 45 mm, about 28 mm to about 50 mm, about 32 mm to about 36 mm, about 32 mm to about 40 mm, about 32 mm to about 45 mm, about 32 mm to about 50 mm, about 36 mm to about 40 mm, about 36 mm to about 45 mm, about 36 mm to about 50 mm, about 40 mm to about 45 mm, about 40 mm to about 50 mm, or about 45 mm to about 50 mm, including increments therein. In some embodiments, the ejection zone is superior to a horizontal line that intersects the inferior aspect of the columella by about 10 mm, about 12 mm, about 16 mm, about 20 mm, about 24 mm, about 28 mm, about 32 mm, about 36 mm, about 40 mm, about 45 mm, or about 50 mm. In some embodiments, the ejection zone is superior to a horizontal line that intersects the inferior aspect of the columella by at least about 10 mm, about 12 mm, about 16 mm, about 20 mm, about 24 mm, about 28 mm, about 32 mm, about 36 mm, about 40 mm, or about 45 mm. In some embodiments, the ejection zone is superior to a horizontal line that intersects the inferior aspect of the columella by at most about 12 mm, about 16 mm, about 20 mm, about 24 mm, about 28 mm, about 32 mm, about 36 mm, about 40 mm, about 45 mm, or about 50 mm. In some embodiments, the ejection zone is superior to a horizontal line that intersects the superior aspect of the inferior turbinate by about 1 mm to about 30 mm. In some embodiments, the ejection zone is superior to a horizontal line that intersects the superior aspect of the inferior turbinate by about 1 mm to about 2 mm, about 1 mm to about 4 mm, about 1 mm to about 6 mm, about 1 mm to about 8 mm, about 1 mm to about 10 mm, about 1 mm to about 12 mm, about 1 mm to about 15 mm, about 1 mm to about 18 mm, about 1 mm to about 21 mm, about 1 mm to about 25 mm, about 1 mm to about 30 mm, about 2 mm to about 4 mm, about 2 mm to about 6 mm, about 2 mm to about 8 mm, about 2 mm to about 10 mm, about 2 mm to about 12 mm, about 2 mm to about 15 mm, about 2 mm to about 18 mm, about 2 mm to about 21 mm, about 2 mm to about 25 mm, about 2 mm to about 30 mm, about 4 mm to about 6 mm, about 4 mm to about 8 mm, about 4 mm to about 10 mm, about 4 mm to about 12 mm, about 4 mm to about 15 mm, about 4 mm to about 18 mm, about 4 mm to about 21 mm, about 4 mm to about 25 mm, about 4 mm to about 30 mm, about 6 mm to about 8 mm, about 6 mm to about 10 mm, about 6 mm to about 12 mm, about 6 mm to about 15 mm, about 6 mm to about 18 mm, about 6 mm to about 21 mm, about 6 mm to about 25 mm, about 6 mm to about 30 mm, about 8 mm to about 10 mm, about 8 mm to about 12 mm, about 8 mm to about 15 mm, about 8 mm to about 18 mm, about 8 mm to about 21 mm, about 8 mm to about 25 mm, about 8 mm to about 30 mm, about 10 mm to about 12 mm, about 10 mm to about 15 mm, about 10 mm to about 18 mm, about 10 mm to about 21 mm, about 10 mm to about 25 mm, about 10 mm to about 30 mm, about 12 mm to about 15 mm, about 12 mm to about 18 mm, about 12 mm to about 21 mm, about 12 mm to about 25 mm, about 12 mm to about 30 mm, about 15 mm to about 18 mm, about 15 mm to about 21 mm, about 15 mm to about 25 mm, about 15 mm to about 30 mm, about 18 mm to about 21 mm, about 18 mm to about 25 mm, about 18 mm to about 30 mm, about 21 mm to about 25 mm, about 21 mm to about 30 mm, or about 25 mm to about 30 mm, including increments therein. In some embodiments, the ejection zone is superior to a horizontal line that intersects the superior aspect of the inferior turbinate by about 1 mm, about 2 mm, about 4 mm, about 6 mm, about 8 mm, about 10 mm, about 12 mm, about 15 mm, about 18 mm, about 21 mm, about 25 mm, or about 30 mm. In some embodiments, the ejection zone is superior to a horizontal line that intersects the superior aspect of the inferior turbinate by at least about 1 mm, about 2 mm, about 4 mm, about 6 mm, about 8 mm, about 10 mm, about 12 mm, about 15 mm, about 18 mm, about 21 mm, or about 25 mm. In some embodiments, the ejection zone is superior to a horizontal line that intersects the superior aspect of the inferior turbinate by at most about 2 mm, about 4 mm, about 6 mm, about 8 mm, about 10 mm, about 12 mm, about 15 mm, about 18 mm, about 21 mm, about 25 mm, or about 30 mm. In some embodiments, the ejection zone is away from the septum by about 0.1 mm to about 3 mm. In some embodiments, the ejection zone is away from the septum by about 0.1 mm to about 0.2 mm, about 0.1 mm to about 0.4 mm, about 0.1 mm to about 0.6 mm, about 0.1 mm to about 0.8 mm, about 0.1 mm to about 1 mm, about 0.1 mm to about 1.25 mm, about 0.1 mm to about 1.5 mm, about 0.1 mm to about 1.75 mm, about 0.1 mm to about 2 mm, about 0.1 mm to about 2.5 mm, about 0.1 mm to about 3 mm, about 0.2 mm to about 0.4 mm, about 0.2 mm to about 0.6 mm, about 0.2 mm to about 0.8 mm, about 0.2 mm to about 1 mm, about 0.2 mm to about 1.25 mm, about 0.2 mm to about 1.5 mm, about 0.2 mm to about 1.75 mm, about 0.2 mm to about 2 mm, about 0.2 mm to about 2.5 mm, about 0.2 mm to about 3 mm, about 0.4 mm to about 0.6 mm, about 0.4 mm to about 0.8 mm, about 0.4 mm to about 1 mm, about 0.4 mm to about 1.25 mm, about 0.4 mm to about 1.5 mm, about 0.4 mm to about 1.75 mm, about 0.4 mm to about 2 mm, about 0.4 mm to about 2.5 mm, about 0.4 mm to about 3 mm, about 0.6 mm to about 0.8 mm, about 0.6 mm to about 1 mm, about 0.6 mm to about 1.25 mm, about 0.6 mm to about 1.5 mm, about 0.6 mm to about 1.75 mm, about 0.6 mm to about 2 mm, about 0.6 mm to about 2.5 mm, about 0.6 mm to about 3 mm, about 0.8 mm to about 1 mm, about 0.8 mm to about 1.25 mm, about 0.8 mm to about 1.5 mm, about 0.8 mm to about 1.75 mm, about 0.8 mm to about 2 mm, about 0.8 mm to about 2.5 mm, about 0.8 mm to about 3 mm, about 1 mm to about 1.25 mm, about 1 mm to about 1.5 mm, about 1 mm to about 1.75 mm, about 1 mm to about 2 mm, about 1 mm to about 2.5 mm, about 1 mm to about 3 mm, about 1.25 mm to about 1.5 mm, about 1.25 mm to about 1.75 mm, about 1.25 mm to about 2 mm, about 1.25 mm to about 2.5 mm, about 1.25 mm to about 3 mm, about 1.5 mm to about 1.75 mm, about 1.5 mm to about 2 mm, about 1.5 mm to about 2.5 mm, about 1.5 mm to about 3 mm, about 1.75 mm to about 2 mm, about 1.75 mm to about 2.5 mm, about 1.75 mm to about 3 mm, about 2 mm to about 2.5 mm, about 2 mm to about 3 mm, or about 2.5 mm to about 3 mm, including increments therein. In some embodiments, the ejection zone is away from the septum by about 0.1 mm, about 0.2 mm, about 0.4 mm, about 0.6 mm, about 0.8 mm, about 1 mm, about 1.25 mm, about 1.5 mm, about 1.75 mm, about 2 mm, about 2.5 mm, or about 3 mm. In some embodiments, the ejection zone is away from the septum by at least about 0.1 mm, about 0.2 mm, about 0.4 mm, about 0.6 mm, about 0.8 mm, about 1 mm, about 1.25 mm, about 1.5 mm, about 1.75 mm, about 2 mm, or about 2.5 mm. In some embodiments, the ejection zone is away from the septum by at most about 0.2 mm, about 0.4 mm, about 0.6 mm, about 0.8 mm, about 1 mm, about 1.25 mm, about 1.5 mm, about 1.75 mm, about 2 mm, about 2.5 mm, or about 3 mm.

By purging a formulation from the olfactory cleft of the patient, the method disclosed herein can control the residence time of a formulation in the olfactory cleft. Where the formulation comprises a therapeutic agent, the method disclosed herein can decrease or terminate the transfer of the therapeutic agent from the olfactory cleft to the central nervous system.

In some embodiments, the method controls the residence time of a formulation in the olfactory cleft. In some embodiments, the purging formulation decreases or terminates the transfer of a therapeutic agent from the olfactory cleft to the central nervous system. In some embodiments, the purging formulation purges a therapeutic formulation from the olfactory cleft.

By purging a therapeutic formulation that has been delivered to the olfactory cleft, the method discloses herein can control a residence time of the therapeutic formulation in the olfactory cleft, and/or decrease or terminate the transfer of a therapeutic agent from the olfactory cleft to the central nervous system.

The duration of time between delivering the therapeutic formulation and delivery of the purging formulation can be selected based on monitoring of a response to the therapeutic formulation. For example, the response can be monitored via a body temperature, a blood pressure measurement, a heart rate measurement, a respiration rate measurement, a blood oxygenation level, electroencephalography (EEG), or observation of pupil dilation. In some embodiments, the duration of time can be at least about 15 seconds, at least about 30 seconds, at least about 45 seconds, at least about 60 seconds, at least about 90 seconds, at least about 120 seconds, at least about 150 seconds, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 40 minutes, at least about 50 minutes, at least about 60 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, or longer than 24 hours.

In some embodiments, the method controls the residence time of the therapeutic formulation in the olfactory cleft. In some embodiments, the purging formulation decreases or terminates the transfer of a therapeutic agent in the therapeutic formulation from the olfactory cleft to the central nervous system.

In some embodiments, the purging formulation is delivered at a velocity sufficient to cause displacement of a substance from the olfactory cleft. In some embodiments, the purging formulation is delivered at a velocity sufficient to cause displacement of the therapeutic formulation from the olfactory cleft. In some embodiments, the velocity at which the purging formulation is ejected from a delivery device is at least about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.2, about 2.4, about 2.6, about 2.8, about 3, about 3.5, about 4, about 4.5, about 5, about 6, about 7, about 8, about 9, about 10, about 12, about 14, about 16, about 18, about 20, about 22, about 24, about 26, about 28, about 30, about 35, about 40, about 45, or about 50 meters per second (m/s). In some embodiments, the velocity at which the purging formulation is ejected from a delivery device is between about 1 m/s and 25 m/s. In some embodiments, the velocity at which the purging formulation is ejected from a delivery device is between about 1 m/s and 20 m/s. In some embodiments, the velocity at which the purging formulation is ejected from a delivery device is between about 3 m/s and 15 m/s.

In some embodiments, a volume of the purging formulation delivered to the olfactory cleft is about 10-20, about 20-30, about 30-40, about 40-50, about 50-60, about 60-70, about 70-80, about 80-90, about 90-100, about 100-120, about 120-140, about 140-160, about 160-180, about 180-200, about 200-250, about 250-300, about 300-350, about 350-400, about 400-450, about 450-500, about 550-600, about 600-650, about 650-700, about 700-750, about 750-800, about 800-850, about 850-900, about 900-950, or about 950-1000 microliters.

In some embodiments, a volume of the purging formulation delivered to the olfactory cleft is less than about 5000 uL. In some embodiments, a volume of the purging formulation delivered to the olfactory cleft is less than about 4500 uL. In some embodiments, a volume of the purging formulation delivered to the olfactory cleft is less than about 4000 uL. In some embodiments, a volume of the purging formulation delivered to the olfactory cleft is less than about 3500 uL. In some embodiments, a volume of the purging formulation delivered to the olfactory cleft is less than about 3000 uL. In some embodiments, a volume of the purging formulation delivered to the olfactory cleft is less than about 2500 uL. In some embodiments, a volume of the purging formulation delivered to the olfactory cleft is less than about 2000 uL. In some embodiments, a volume of the purging formulation delivered to the olfactory cleft is less than about 1500 uL. In some embodiments, a volume of the purging formulation delivered to the olfactory cleft is less than about 1000 uL. In some embodiments, a volume of the purging formulation delivered to the olfactory cleft is less than about 900 uL. In some embodiments, a volume of the purging formulation delivered to the olfactory cleft is less than about 800 uL. In some embodiments, a volume of the purging formulation delivered to the olfactory cleft is less than about 700 uL. In some embodiments, a volume of the purging formulation delivered to the olfactory cleft is less than about 600 uL. In some embodiments, a volume of the purging formulation delivered to the olfactory cleft is less than about 500 uL. In some embodiments, a volume of the purging formulation delivered to the olfactory cleft is less than about 400 uL. In some embodiments, a volume of the purging formulation delivered to the olfactory cleft is less than about 300 uL. In some embodiments, a volume of the purging formulation delivered to the olfactory cleft is less than about 200 uL. In some embodiments, a volume of the purging formulation delivered to the olfactory cleft is less than about 100 uL. In some embodiments, a volume of the purging formulation delivered to the olfactory cleft is less than about 90 uL. In some embodiments, a volume of the purging formulation delivered to the olfactory cleft is less than about 80 uL. In some embodiments, a volume of the purging formulation delivered to the olfactory cleft is less than about 70 uL. In some embodiments, a volume of the purging formulation delivered to the olfactory cleft is less than about 60 uL. In some embodiments, a volume of the purging formulation delivered to the olfactory cleft is less than about 50 uL. In some embodiments, a volume of the purging formulation delivered to the olfactory cleft is less than about 40 uL. In some embodiments, a volume of the purging formulation delivered to the olfactory cleft is less than about 30 uL. In some embodiments, a volume of the purging formulation delivered to the olfactory cleft is less than about 20 uL. In some embodiments, a volume of the purging formulation delivered to the olfactory cleft is less than about 10 uL.

In some embodiments, the purging formulation has a viscosity of about 0.5-0.6, about 0.6-0.7, about 0.7-0.8, about 0.8-0.9, about 0.9-1, about 1-2, about 2-3, about 3-4, about 4-5, about 5-6, about 6-7, about 7-8, about 8-9, about 9-10, about 10-20, about 20-30, about 30-40, about 40-50, about 50-60, about 60-70, about 70-80, about 80-90, about 90-100, about 100-120, about 120-140, about 140-160, about 160-180, about 180-200, about 200-250, about 250-300, about 300-350, about 350-400, about 400-450, about 450-500, about 550-600, about 600-650, about 650-700, about 700-750, about 750-800, about 800-850, about 850-900, about 900-950, or about 950-1000 centipoise (cP). In some embodiments, the purging formulation has a viscosity of between about 0.5 cP and 50 cP. In some embodiments, the purging formulation has a viscosity of between about 0.5 cP and 25 cP.

Both the purging formulation and the therapeutic formulation can be ejected from a delivery device. The volume of the purging formulation ejected may be about the same, lower than, or higher than the therapeutic formulation ejected. In some embodiments, the volume of the purging formulation ejected is about the same as the volume of therapeutic formulation ejected. In some embodiments, the volume of the purging formulation ejected is lower than the volume of therapeutic formulation ejected. In some embodiments, the volume of the purging formulation ejected is higher than the volume of therapeutic formulation ejected. In some embodiments, the volume of the purging formulation ejected is not more than about 4-fold the volume of the therapeutic formulation ejected. In some embodiments, the volume of the purging formulation ejected is not more than about 3.5-fold the volume of the therapeutic formulation ejected. In some embodiments, the volume of the purging formulation ejected is not more than about 3-fold the volume of the therapeutic formulation ejected. In some embodiments, the volume of the purging formulation ejected is not more than about 2.5-fold the volume of the therapeutic formulation ejected. In some embodiments, the volume of the purging formulation ejected is not more than about 2-fold the volume of the therapeutic formulation ejected. In some embodiments, the volume of the purging formulation ejected is not more than about 1.5-fold the volume of the therapeutic formulation ejected. In some embodiments, the volume of the purging formulation ejected is not more than about the volume of the therapeutic formulation ejected.

Where the therapeutic formulation is ejected from a delivery device, a percentage of the ejected therapeutic formulation may be delivered to the olfactory cleft. After the duration of time, a percentage of the therapeutic formulation delivered to the olfactory cleft may remain at the olfactory cleft. The purging formulation can purge the remaining therapeutic formulation from the olfactory cleft. In some embodiments, the purging formulation purges at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the remaining therapeutic formulation from the olfactory cleft.

In some embodiments, the purging formulation forms a bolus within the olfactory cleft. In some embodiments, the purging formulation forms a capillary bridge at a base of the olfactory cleft. In some embodiments, the purging formulation dilutes a bolus already within the olfactory cleft. In some embodiments, the purging formulation physically displaces a substance from the olfactory cleft. In some embodiments, the substance comprises a therapeutic agent. In some embodiments, the purging formulation exerts a pressure on a substance within the olfactory cleft to purge the substance from the olfactory cleft. In some embodiments, the purging formulation draws a substance from within the olfactory cleft into the purging formulation. In some embodiments, the purging formulation has a sufficiently low osmolality to draw a substance from within the olfactory cleft into the purging formulation. In some embodiments, the purging formulation purges at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of a substance from the olfactory cleft.

The therapeutic formulation can comprise a therapeutic agent. The therapeutic agent can be any agent that can treat or prevent a disease, condition, or disorder, including but not limited to proteins, peptides, antibodies, vaccines, biologics, nucleic acids, vectors, viral vectors, small molecules, hormones, lipid nanoparticles, nanoparticles, etc.

In some embodiments, the substance comprises a therapeutic agent.

In some embodiments, the purging formulation impedes transfer of an intranasal therapeutic agent to the olfactory cleft by reducing or eliminating contact between the therapeutic agent and a tissue within the olfactory cleft. In some embodiments, the purging formulation reduces a rate of drug transmission via the olfactory cleft. In some embodiments, the purging formulation is configured to cause an osmotic pressure sufficient to reverse a direction of a drug flow through a tissue of the olfactory cleft.

In some embodiments, the purging formulation has an identical composition to that of the therapeutic formulation, except for the absence of a therapeutic agent.

In some embodiments, the purging formulation comprises water. In some embodiments, the purging formulation comprises one or more surfactants. In some embodiments, the purging formulation comprises one or more flavoring agents. In some embodiments, the purging formulation is configured to neutralize a therapeutic agent. In some embodiments, the purging formulation has a pH sufficient to cause degradation of a therapeutic agent.

In some embodiments, the purging formulation comprises an enzyme configured to cause degradation of a therapeutic agent. In some embodiments, the purging formulation comprises a therapeutic agent configured for absorption into a tissue in the respiratory nasal cavity or the gastrointestinal tract. In some embodiments, the purging formulation is sterilized prior to delivery to the olfactory cleft.

In some embodiments, the purging formulation is delivered as a single volume. In some embodiments, the purging formulation is delivered as multiple separate volumes. In some embodiments, a first volume of purging formulation is delivered to the olfactory cleft and a second volume of purging formulation is delivered to the olfactory cleft after waiting for a time period. In some embodiments, multiple sequential volumes of the purging formulation are delivered.

In some embodiments, the purging formulation is delivered via a designated left or right applicator configured for delivery to a left or right olfactory cleft. In some embodiments, the purging formulation is delivered selectively to a left or a right olfactory cleft. In some embodiments, the purging formulation is delivered selectively to a left or a right olfactory cleft and a second purging formulation is delivered selectively to the other olfactory cleft.

In some embodiments, the purging formulation has a density which is less than a density of the therapeutic formulation. In some embodiments, the purging formulation displaces the therapeutic formulation from the olfactory cleft due to relative buoyancy of the purging formulation in comparison to the therapeutic formulation. Without limiting the present invention to any particular theory or mechanism, the less dense purging formulation may rise to the top of a bolus formed by the two formulations so as to apply downward pressure on the therapeutic formulation, thereby separating or partially separating the therapeutic formulation from a surface of the olfactory cleft, or removing the therapeutic formulation from the olfactory cleft. In some embodiments, the therapeutic formulation has a density which is at least about 1, at least about 2, at least about 3, at least about 4, at least about 5, at least about 6, at least about 7, at least about 8, at least about 9, at least about 10, at least about 12, at least about 14, at least about 16, at least about 18, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 percent greater than a density of the purging formulation. In some embodiments the therapeutic formulation and the purging formulation are miscible at use temperatures (i.e. room temperature or body temperature). In some embodiments the therapeutic formulation and the purging formulation are immiscible at use temperatures (i.e. room temperature or body temperature).

In some embodiments, the purging formulation effects a physical property of the therapeutic formulation after contacting the therapeutic formulation, thereby facilitating removal of the therapeutic formulation, wherein the physical property is selected from the group consisting of: viscosity, surface tension, density, and adhesion. In some embodiments, the purging formulation replaces an irritant in the olfactory cleft with a therapeutic agent configured to provide a therapeutic effect to the olfactory cleft. In some embodiments, the purging formulation comprises a therapeutic agent which is different from a therapeutic agent of the therapeutic formulation. In some embodiments, the purging formulation comprises a coloring agent. In some embodiments, the purging formulation comprises a coloring agent which changes color after contact with the therapeutic formulation, thereby serving as an indicator of contact with the therapeutic formulation or removal of the therapeutic formulation.

In some embodiments, the method additionally comprises delivery of a second therapeutic formulation to the olfactory cleft after the therapeutic formulation is purged from the olfactory cleft via the purging formulation. In some embodiments, the therapeutic formulation forms a bolus within the olfactory cleft or within another intranasal region. In some embodiments, the therapeutic formulation forms a capillary bridge at a base of the olfactory cleft. In some embodiments, the therapeutic formulation comprises one or more flavoring agents or odorants. In some embodiments, purging of the therapeutic formulation releases a flavoring agent or odorant to be detected by the patient, thereby signaling successful purging of the therapeutic formulation.

In some embodiments, the therapeutic formulation and the purging formulation are delivered via ejection from a device at a same depth within the nasal cavity. In some embodiments, the therapeutic formulation and the purging formulation are delivered via ejection from a device at different depths within the nasal cavity. In some embodiments, the therapeutic formulation and the purging formulation are delivered via ejection from a device at a same angle within the nasal cavity. In some embodiments, the therapeutic formulation and the purging formulation are delivered via ejection from a device at different angles within the nasal cavity.

In some embodiments, the therapeutic formulation and the purging formulation are delivered from a single delivery device without a need to reload the device between delivery of the therapeutic formulation and delivery of the purging formulation. In some embodiments, the therapeutic formulation and the purging formulation are delivered via a single dual-chamber device. In some embodiments, the therapeutic formulation and the purging formulation are delivered via a device configured to sequentially deliver the therapeutic formulation from a first primary drug container and the purging formulation from a secondary drug container. In some embodiments, the therapeutic formulation and the purging formulation are delivered via a device containing the therapeutic formulation and the purging formulation in a single primary drug container. In some embodiments, the therapeutic formulation and the purging formulation are delivered via a device including a timer 704 for measurement of a predetermined residence time between delivery of the therapeutic formulation and delivery of the purging formulation.

In some embodiments, the duration of time is selected based on monitoring of a response to the therapeutic formulation.

In some embodiments, a residence time between delivery of the therapeutic formulation and delivery of the purging formulation is selected based on a monitored biomarker. In some embodiments, a residence time between delivery of the therapeutic formulation and delivery of the purging formulation is selected based on a monitored biomarker selected from the group consisting of: a body temperature, a blood pressure measurement, a heart rate measurement, a respiration rate measurement, a blood oxygenation level, electroencephalography (EEG), and observation of pupil dilation.

In another aspect, disclosed herein is a method of purging left and right olfactory clefts of a patient, the method comprising: providing a left purging formulation configured to purge the left olfactory cleft; providing a right purging formulation configured to purge the right olfactory cleft; delivering the left purging formulation to the left olfactory cleft, thereby purging the left olfactory cleft; and delivering the right purging formulation to the right olfactory cleft, thereby purging the right olfactory cleft. In one aspect, provided herein is a method of purging left and right olfactory clefts of a patient. The method can comprise providing a left purging formulation. The left purging formulation can be configured to purge the left olfactory cleft. The method can comprise providing a right purging formulation. The right purging formulation can be configured to purge the right olfactory cleft. The method can comprise delivering the left purging formulation to the left olfactory cleft. The method can comprise delivering the right purging formulation to the right olfactory cleft, thereby purging the right olfactory cleft.

In some embodiments, the left purging formulation and the right purging formulation are identical. In some embodiments, the left purging formulation and the right purging formulation are different. In some embodiments, the left purging formulation and the right purging formulation are delivered at the same time. In some embodiments, the left purging formulation and the right purging formulation are delivered at different times. In some embodiments, the left purging formulation and the right purging formulation are delivered in equal volumes. In some embodiments, the left purging formulation and the right purging formulation are delivered in different volumes.

In another aspect, disclosed herein is a method of controlling delivery of a therapeutic formulation via an olfactory cleft of a patient, the method comprising: delivering a first volume of a therapeutic formulation to the olfactory cleft; monitoring a response to the therapeutic formulation; and delivering one or more additional volumes of the therapeutic formulation to the olfactory cleft based on the monitored response to the therapeutic formulation. In some embodiments, the response is monitored via a body temperature, a blood pressure measurement, a heart rate measurement, a respiration rate measurement, a blood oxygenation level, electroencephalography (EEG), or observation of pupil dilation.

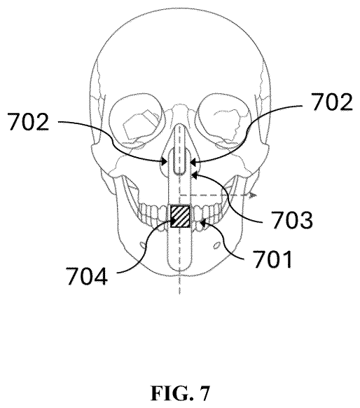

Referring to FIG. 7. The method disclosed herein can comprise delivering the purging formulation via a device comprising a housing 701 defining a first insertable portion 702 and a second insertable portion 702, and an actuator 703 which delivers the purging formulation from the either or both of the insertable portions 702 when the device is actuated. Each of the insertable portions can be for insertion into a nasal channel of the patient. Upon insertion of at least one insertable portion into the nasal channel of the patient, the at least one insertable portion engages tissue within the nasal channel to open or expand an internal nasal valve of the patient thereby positioning at least one of the insertable portions for delivery of the purging formulation to the olfactory region of the patient.

In some embodiments, the purging formulation is delivered via a device comprising: a housing defining first and second insertable portions, each for insertion into a nasal channel of the subject, wherein, upon insertion of the first insertable portion into the nasal channel of the subject, the at least one insertable portion engages tissue within the nasal channel to open or expand an internal nasal valve of the subject thereby positioning at least one of the insertable portions for delivery of the purging formulation to the olfactory region of the subject; and an actuator which delivers the purging formulation from the either or both of the insertable portions when the device is actuated.

### II. Devices

In yet another aspect, disclosed herein is a device for purging an olfactory cleft of a patient, the device comprising: a housing comprising an insertable portion comprising a distal end, and a proximal end; and a subject-engaging portion which engages a columella region of the subject to seat the distal end of the insertable portion within an ejection zone of a nasal channel of the subject; wherein the device is configured to deliver a purging formulation to the olfactory cleft of the patient, thereby purging the olfactory cleft.

As used herein, the term “distal” generally refers to the direction towards the end of the device configured to dispense fluid and the term “proximal” generally refers to the direction towards the end of the device furthest from a fluid dispensing tip.

In a further aspect, disclosed herein is a device for controlled delivery of a therapeutic formulation via an olfactory cleft of a patient, the device comprising: a first chamber configured to hold the therapeutic formulation; a second chamber configured to hold a purging formulation for purging the therapeutic formulation from the olfactory cleft; and a delivery mechanism configured to sequentially deliver the therapeutic formulation and the purging formulation to the olfactory cleft. In some embodiments, the device additionally comprises a dispensing tip configured to allow for precision delivery of the therapeutic formulation and the purging formulation to the olfactory cleft. In some embodiments, the device is configured to eject each of the therapeutic formulation and the purging formulation as sequential laminar jets.

### III. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−10% or less from the specified value, insofar such variations are appropriate to perform in the present disclosure. It is to be understood that the value to which the modifier “about” or “approximately” refers is itself also specifically disclosed.

The term “nasal cavity” comprises a vestibule, respiratory region, olfactory cleft, and a nasopharynx.

Turbinates: These refer to superior turbinate, middle turbinate, or inferior turbinate, or a combination thereof.

Nasal Cavity: This is the large, air-filled space behind the nose, where air passes on its way to the throat during inhalation.

Internal Nasal Valve: This is the narrowest part of the nasal airway, located just beyond the nostril. It's formed by the edge of the nasal septum, the upper lateral cartilage, and the floor of the nose. The internal nasal valve plays a critical role in regulating airflow through the nose. The area of interest is superior (above) to this structure.

Nasal Septum: This is the thin wall of bone and cartilage that separates the right and left nostrils. It forms the medial (towards the middle) boundary of the region of interest.

Lateral Nasal Wall: This is the side wall of the nasal cavity, which is opposite to the nasal septum. It's a complex structure that includes the turbinates (long, curled bones that protrude into the nasal cavity) and the meatuses (grooves or channels between the turbinates). The lateral nasal wall forms the lateral (towards the side) boundary of the region of interest.

Middle and Superior Meatuses: These are the spaces within the nasal cavity located between the turbinates. The middle meatus is located beneath the middle turbinate and above the inferior turbinate, and the superior meatus is located beneath the superior turbinate. The region of interest encompasses parts of these spaces.

Nostrils (External Nares): These are the two openings of the nose where air enters.

Nasal Vestibule: The nasal vestibule is the most anterior part of the nasal cavity, just inside the nostrils. It is the area of the nose that protrudes outside the face predominantly. This area is lined with skin and contains hair follicles, and it acts as the initial filtering and warming area for inhaled air before it moves deeper into the nasal cavity. The nasal vestibule extends posteriorly to the nasal valve, which is the narrowest part of the nasal airway and located just beyond the nostril.

Nasal Septum: This is a thin wall made of bone and cartilage that separates the left and right sides of the nasal cavity.

Turbinates (Nasal Conchae): These are three pairs of bony projections (inferior, middle, and superior) covered in mucous membrane that protrude into the nasal cavity from the lateral walls. They increase the surface area of the nasal cavity, aiding in the warming, humidification, and filtration of inhaled air.

Meatuses: These are the spaces located between the turbinates. Each turbinate has a corresponding meatus underneath it (i.e., inferior, middle, and superior meatus).

Olfactory Region: This is a small area located at the top of the nasal cavity, where the sense of smell is located. In some embodiments, the olfactory region is an olfactory cleft.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

Example 1: Delivery of a therapeutic formulation to an olfactory cleft of a patient for a duration selected based on monitoring of patient biometrics.

Step 1: A patient is prescribed a dose of a therapeutic formulation for the treatment of a diagnosed condition.

Step 2: An intranasal delivery device is provided, the device having separate reservoirs containing the therapeutic formulation and a purging formulation.

Step 3: The delivery device is positioned with a dispensing tip aimed at an olfactory cleft of the patient, and a dose of the therapeutic formulation is dispensed from the dispensing tip such that it is delivered to the olfactory cleft and forms a bolus within the olfactory cleft.

Step 4: The bolus of the therapeutic formulation is allowed to remain within the olfactory cleft for a duration of time to allow for delivery of a therapeutic agent to the central nervous system via the olfactory cleft.

Step 5: One or more biometrics of the patient are monitored to evaluate effects of the therapeutic agent on the patient.

Step 6: A knockout time for the dose of the therapeutic formulation is determined based on the monitored patient biometrics.

Step 7: The delivery device is positioned with a dispensing tip aimed at the olfactory cleft of the patient, and a volume of the purging formulation is dispensed from the dispensing tip such that it is delivered to the olfactory cleft and displaces at least a portion of the bolus of the therapeutic formulation from the olfactory cleft, thereby decreasing or stopping delivery of the therapeutic agent to the central nervous system via the olfactory cleft.

### Example 2: Delivery to and Purging of a Formulation from the Olfactory of a Model Nasal Cavity

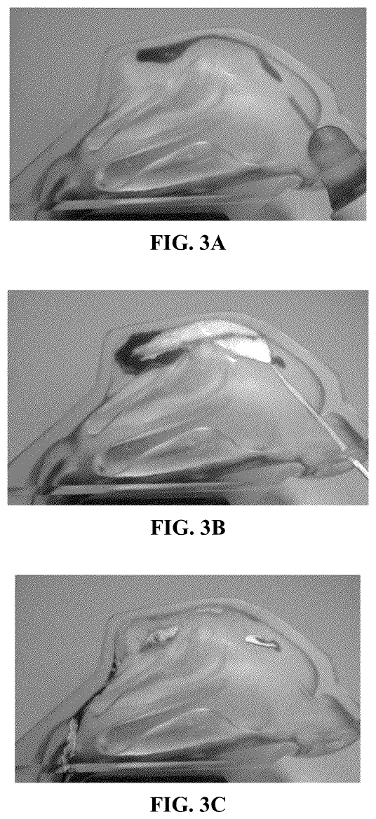

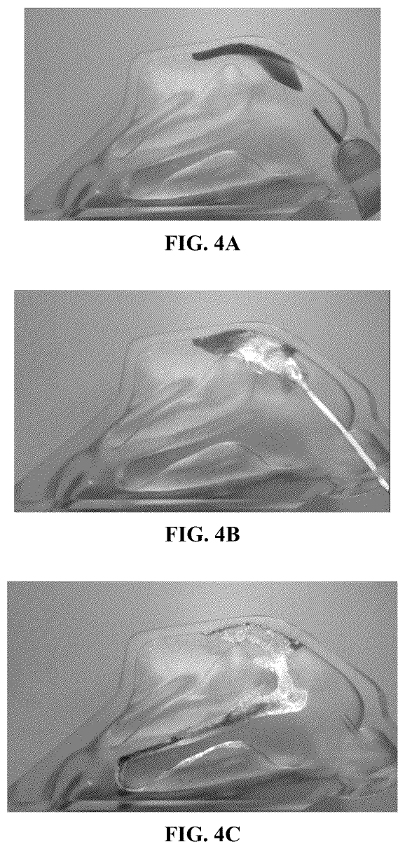

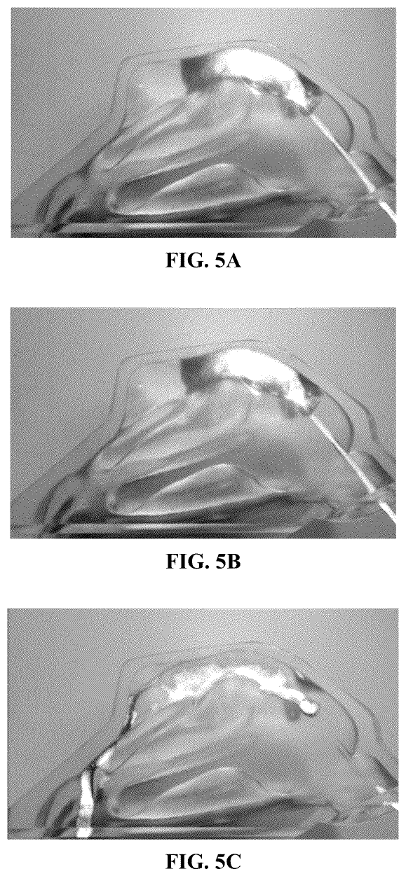

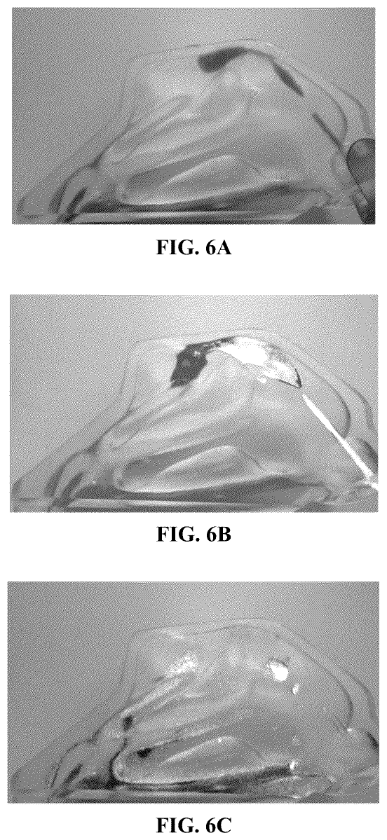

100 uL of a primary dose was deposited at the olfactory region of a model nasal cavity. 200 uL of a secondary dose was administered within 30 seconds of delivery of the primary dose. Deposition was captured with a high-speed camera. The devices used in ejecting the doses were inserted at a depth of 32.5 mm and at an angle of 35 degrees. Four tests were performed with specific viscosities for the doses. Images captured by the high-speed camera are shown in FIGS. 3A-3C, FIGS. 4A-4C, FIGS. 5A-5C, and FIGS. 6A-6C. In Test 1, both the primary and the secondary doses had a viscosity of 1 cP (Table 1, FIGS. 3A-3C). In Test 2, both the primary and the secondary doses had a viscosity of 20 cP (Table 2, FIGS. 4A-4C). In Test 3, the primary dose had a viscosity of 20 cP and the secondary dose had a viscosity of 1 cP (Table 3, FIGS. 5A-5C). In Test 4, the primary dose had a viscosity of 1 cP and the secondary dose had a viscosity of 20 cP (Table 4, FIGS. 6A-6C).

TABLE 1
Ejection parameters for Test 1

| | Output | | |
|---|---|---|---|
| Dose | Volume (ul) | Output Velocity (m/s) | Viscosity (cP) |
| Primary Dose | 100 ul | 2.5 m/s | 1 cP |
| Secondary Dose | 200 ul | 5 m/s | 1 cP |

TABLE 2
Ejection parameters for Test 2

| | Output | | |
|---|---|---|---|
| Dose | Volume (ul) | Output Velocity (m/s) | Viscosity (cP) |
| Primary Dose | 100 ul | 6 m/s | 20 cP |
| Secondary Dose | 200 ul | 8 m/s | 20 cP |

TABLE 3
Ejection parameters for Test 3

| | Output | | |
|---|---|---|---|
| Dose | Volume (ul) | Output Velocity (m/s) | Viscosity (cP) |
| Primary Dose | 100 ul | 6 m/s | 20 cP |
| Secondary Dose | 200 ul | 9 m/s | 1 cP |

TABLE 4
Ejection parameters for Test 4

| | Output | | |
|---|---|---|---|
| Dose | Volume (ul) | Output Velocity (m/s) | Viscosity (cP) |
| Primary Dose | 100 ul | 2.5 m/s | 1 cP |
| Secondary Dose | 200 ul | 8 m/s | 20 cP |

### Enumerated Embodiments

Enumerated Embodiment 1. A method of delivery of a therapeutic formulation via an olfactory cleft of a patient, the method comprising:
   a. delivering the therapeutic formulation to the olfactory cleft;
   b. waiting for a duration of time; and
   c. delivering a purging formulation to the olfactory cleft, thereby purging the therapeutic formulation from the olfactory cleft.
Enumerated Embodiment 2. The method of any one of the enumerated embodiments, wherein the purging formulation is ejected from a delivery device as a liquid jet.
Enumerated Embodiment 3. The method of any one of the enumerated embodiments, wherein the purging formulation is ejected from a delivery device as a laminar flow.
Enumerated Embodiment 4. The method of any one of the enumerated embodiments, wherein the purging formulation is ejected from an ejection zone in a nasal cavity of the subject, wherein the ejection zone is:
   a. 0 mm to 30 mm superior to a horizontal line that intersects the anterior aspect of the internal nasal valve, and
   b. 0 mm to 20 mm anterior to an inclined line that intersects the anterior aspect of the middle turbinate and the posterior aspect of the vestibule.
Enumerated Embodiment 5. The method of any one of the enumerated embodiments, wherein the ejection zone is further:
   a. 0 mm to 40 mm inferior to a horizontal line that is parallel to the inferior aspect of the olfactory cleft,
   b. 0 mm to 20 mm posterior to the internal nasal dorsum,
   c. 10 mm to 50 mm superior to a horizontal line that intersects the inferior aspect of the columella,
   d. 0 mm to 30 mm superior to a horizontal line that intersects the superior aspect of the inferior turbinate,
   e. 0 mm to 3 mm from the septum, or
   f. any combination thereof.
Enumerated Embodiment 6. The method of any one of the enumerated embodiments, wherein the purging formulation precisely targets the olfactory cleft.
Enumerated Embodiment 7. The method of any one of the enumerated embodiments, wherein the purging formulation is delivered from a position sufficiently deep within the nasal cavity to allow for precise targeting of the olfactory cleft or another intranasal region.
Enumerated Embodiment 8. The method of any one of the enumerated embodiments, wherein the method controls the residence time of a formulation in the olfactory cleft.
Enumerated Embodiment 9. The method of any one of the enumerated embodiments, wherein the purging formulation decreases or terminates the transfer of a therapeutic agent from the olfactory cleft to the central nervous system.
Enumerated Embodiment 10. The method of any one of the enumerated embodiments, wherein the purging formulation is delivered at a velocity sufficient to cause displacement of the therapeutic formulation from the olfactory cleft.
Enumerated Embodiment 11. The method of any one of the enumerated embodiments, wherein the purging formulation is delivered at a velocity of between about 1 m/s and 20 m/s.
Enumerated Embodiment 12. The method of any one of the enumerated embodiments, wherein the purging formulation is delivered at a velocity of between about 3 m/s and 15 m/s.
Enumerated Embodiment 13. The method of any one of the enumerated embodiments, wherein the purging formulation has a viscosity of between 0.5 cP and 50 cP.
Enumerated Embodiment 14. The method of any one of the enumerated embodiments, wherein the purging formulation has a viscosity of between 0.5 cP and 25 cP.
Enumerated Embodiment 15. The method of any one of the enumerated embodiments wherein the purging formulation and the therapeutic formulation are delivered via ejection from a delivery device, and wherein the volume of the purging formulation ejected is not more than 2-fold the volume of the therapeutic formulation.
Enumerated Embodiment 16. The method of any one of the enumerated embodiments, wherein the purging formulation forms a bolus within the olfactory cleft.
Enumerated Embodiment 17. The method of any one of the enumerated embodiments, wherein the purging formulation forms a capillary bridge at a base of the olfactory cleft.
Enumerated Embodiment 18. The method of any one of the enumerated embodiments, wherein the purging formulation dilutes a bolus already within the olfactory cleft.
Enumerated Embodiment 19. The method of any one of the enumerated embodiments, wherein the purging formulation physically displaces a substance from the olfactory cleft.
Enumerated Embodiment 20. The method of any one of the enumerated embodiments, wherein the purging formulation exerts a pressure on a substance within the olfactory cleft to purge the substance from the olfactory cleft.
Enumerated Embodiment 21. The method of any one of the enumerated embodiments, wherein the purging formulation draws a substance from within the olfactory cleft into the purging formulation. Enumerated Embodiment 22. The method of any one of the enumerated embodiments, wherein the purging formulation has a sufficiently low osmolality to draw a substance from within the olfactory cleft into the purging formulation.
Enumerated Embodiment 23. The method of any one of the enumerated embodiments, wherein the substance comprises a therapeutic agent.
Enumerated Embodiment 24. The method of any one of the enumerated embodiments, wherein the purging formulation impedes transfer of an intranasal therapeutic agent to the olfactory cleft by reducing or eliminating contact between the therapeutic agent and a tissue within the olfactory cleft. Enumerated Embodiment 25. The method of any one of the enumerated embodiments, wherein the purging formulation reduces a rate of drug transmission via the olfactory cleft.
Enumerated Embodiment 26. The method of any one of the enumerated embodiments, wherein the purging formulation is configured to cause an osmotic pressure sufficient to reverse a direction of a drug flow through a tissue of the olfactory cleft.
Enumerated Embodiment 27. The method of any one of the enumerated embodiments, wherein the purging formulation is configured to neutralize a therapeutic agent.
Enumerated Embodiment 28. The method of any one of the enumerated embodiments, wherein the purging formulation has a pH sufficient to cause degradation of a therapeutic agent.
Enumerated Embodiment 29. The method of any one of the enumerated embodiments, wherein the purging formulation comprises an enzyme configured to cause degradation of a therapeutic agent.
Enumerated Embodiment 30. The method of any one of the enumerated embodiments, wherein the purging formulation comprises a therapeutic agent configured for absorption into a tissue in the respiratory nasal cavity or the gastrointestinal tract.
Enumerated Embodiment 31. The method of any one of the enumerated embodiments, wherein the purging formulation is delivered as a single volume.
Enumerated Embodiment 32. The method of any one of the enumerated embodiments, wherein the purging formulation is delivered as multiple separate volumes.
Enumerated Embodiment 33. The method of any one of the enumerated embodiments, wherein a first volume of purging formulation is delivered to the olfactory cleft and a second volume of purging formulation is delivered to the olfactory cleft after a time period.
Enumerated Embodiment 34. The method of any one of the enumerated embodiments, wherein the purging formulation is delivered via a designated left or right applicator configured for delivery to a left or right olfactory cleft.
Enumerated Embodiment 35. The method of any one of the enumerated embodiments, wherein the purging formulation is delivered selectively to a left or a right olfactory cleft.
Enumerated Embodiment 36. The method of any one of the enumerated embodiments, wherein the purging formulation is delivered selectively to a left or a right olfactory cleft and a second purging formulation is delivered selectively to the other olfactory cleft.
Enumerated Embodiment 37. The method of any one of the enumerated embodiments, wherein the purging formulation has a density less than a density of the therapeutic formulation.
Enumerated Embodiment 38. The method of any one of the enumerated embodiments, wherein the purging formulation displaces the therapeutic formulation from the olfactory cleft due to relative buoyancy of the purging formulation in comparison to the therapeutic formulation.
Enumerated Embodiment 39. The method of any one of the enumerated embodiments, wherein the duration of time is selected based on monitoring of a response to the therapeutic formulation.
Enumerated Embodiment 40. The method of any one of the enumerated embodiments, wherein the purging formulation has an identical composition to that of the therapeutic formulation, except for the absence of a therapeutic agent.
Enumerated Embodiment 41. The method of any one of the enumerated embodiments, wherein the purging formulation effects a physical property of the therapeutic formulation after contacting the therapeutic formulation, thereby facilitating removal of the therapeutic formulation, wherein the physical property is selected from the group consisting of: viscosity, surface tension, density, and adhesion.
Enumerated Embodiment 42. The method of any one of the enumerated embodiments, wherein the purging formulation comprises a therapeutic agent which is different from a therapeutic agent of the therapeutic formulation.
Enumerated Embodiment 43. The method of any one of the enumerated embodiments, wherein the purging formulation comprises a coloring agent which changes color after contact with the therapeutic formulation, thereby serving as an indicator of contact with the therapeutic formulation or removal of the therapeutic formulation.
Enumerated Embodiment 44. The method of any one of the enumerated embodiments, additionally comprising delivery of a second therapeutic formulation to the olfactory cleft after the therapeutic formulation is purged from the olfactory cleft via the purging formulation.
Enumerated Embodiment 45. The method of any one of the enumerated embodiments, wherein the therapeutic formulation comprises one or more flavoring agents or odorants.
Enumerated Embodiment 46. The method of any one of the enumerated embodiments, wherein purging of the therapeutic formulation releases the one or more flavoring agents or odorants detectable by the patient.
Enumerated Embodiment 47. The method of any one of the enumerated embodiments, wherein the therapeutic formulation and the purging formulation are delivered via ejection from a device at a same depth within the nasal cavity.
Enumerated Embodiment 48. The method of any one of the enumerated embodiments, wherein the therapeutic formulation and the purging formulation are delivered via ejection from a device at different depths within the nasal cavity.
Enumerated Embodiment 49. The method of any one of the enumerated embodiments, wherein the therapeutic formulation and the purging formulation are delivered via ejection from a device at a same angle within the nasal cavity.
Enumerated Embodiment 50. The method of any one of the enumerated embodiments, wherein the therapeutic formulation and the purging formulation are delivered via ejection from a device at different angles within the nasal cavity.
Enumerated Embodiment 51. The method of any one of the enumerated embodiments, wherein the therapeutic formulation and the purging formulation are delivered from a single delivery device without a need to reload the device between delivery of the therapeutic formulation and delivery of the purging formulation.
Enumerated Embodiment 52. The method of any one of the enumerated embodiments, wherein the therapeutic formulation and the purging formulation are delivered via a single dual-chamber device. Enumerated Embodiment 53. The method of any one of the enumerated embodiments, wherein the therapeutic formulation and the purging formulation are delivered via a device configured to sequentially deliver the therapeutic formulation from a first primary drug container and the purging formulation from a secondary drug container.
Enumerated Embodiment 54. The method of any one of the enumerated embodiments, wherein the therapeutic formulation and the purging formulation are delivered via a device containing the therapeutic formulation and the purging formulation in a single primary drug container.
Enumerated Embodiment 55. The method of any one of the enumerated embodiments, wherein the therapeutic formulation and the purging formulation are delivered via a device including a timer for measurement of a predetermined residence time between delivery of the therapeutic formulation and delivery of the purging formulation.
Enumerated Embodiment 56. The method of any one of the enumerated embodiments, wherein a residence time between delivery of the therapeutic formulation and delivery of the purging formulation is selected based on a monitored biomarker.
Enumerated Embodiment 57. The method of any one of the enumerated embodiments, wherein a residence time between delivery of the therapeutic formulation and delivery of the purging formulation is selected based on a monitored biomarker selected from the group consisting of: a body temperature, a blood pressure measurement, a heart rate measurement, a respiration rate measurement, a blood oxygenation level, electroencephalography (EEG), and observation of pupil dilation.
Enumerated Embodiment 58. A method of purging an olfactory cleft of a patient, the method comprising:
   a. providing a purging formulation configured to purge the olfactory cleft; and
   b. delivering the purging formulation to the olfactory cleft, thereby purging the olfactory cleft.
Enumerated Embodiment 59. The method of any one of the enumerated embodiments, wherein the purging formulation purges a therapeutic formulation from the olfactory cleft.
Enumerated Embodiment 60. The method of any one of the enumerated embodiments, wherein the purging formulation replaces an irritant in the olfactory cleft with a therapeutic agent configured to provide a therapeutic effect to the olfactory cleft.
Enumerated Embodiment 61. The method of any one of the enumerated embodiments, wherein the purging formulation is ejected from a delivery device as a liquid jet.
Enumerated Embodiment 62. The method of any one of the enumerated embodiments, wherein the purging formulation precisely targets the olfactory cleft.
Enumerated Embodiment 63. The method of any one of the enumerated embodiments, wherein the purging formulation is delivered from a position sufficiently deep within the nasal cavity to allow for precise targeting of the olfactory cleft or another intranasal region.
Enumerated Embodiment 64. The method of any one of the enumerated embodiments, wherein the method controls the residence time of a formulation in the olfactory cleft.
Enumerated Embodiment 65. The method of any one of the enumerated embodiments, wherein the purging formulation decreases or terminates the transfer of a therapeutic agent from the olfactory cleft to the central nervous system.
Enumerated Embodiment 66. The method of any one of the enumerated embodiments, wherein the purging formulation purges a therapeutic formulation from the olfactory cleft.
Enumerated Embodiment 67. The method of any one of the enumerated embodiments, wherein the purging formulation is delivered at a velocity sufficient to cause displacement of a substance from the olfactory cleft.
Enumerated Embodiment 68. The method of any one of the enumerated embodiments, wherein the velocity at which the purging formulation is ejected from a delivery device is at least about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.2, about 2.4, about 2.6, about 2.8, about 3, about 3.5, about 4, about 4.5, about 5, about 6, about 7, about 8, about 9, about 10, about 12, about 14, about 16, about 18, about 20, about 22, about 24, about 26, about 28, about 30, about 35, about 40, about 45, or about 50 meters per second (m/s). Enumerated Embodiment 69. The method of any one of the enumerated embodiments, wherein the velocity at which the purging formulation is ejected from a delivery device is between about 1-25 m/s.
Enumerated Embodiment 70. The method of any one of the enumerated embodiments, wherein a volume of the purging formulation delivered to the olfactory cleft is about 10-20, about 20-30, about 30-40, about 40-50, about 50-60, about 60-70, about 70-80, about 80-90, about 90-100, about 100-120, about 120-140, about 140-160, about 160-180, about 180-200, about 200-250, about 250-300, about 300-350, about 350-400, about 400-450, about 450-500, about 550-600, about 600-650, about 650-700, about 700-750, about 750-800, about 800-850, about 850-900, about 900-950, or about 950-1000 microliters.
Enumerated Embodiment 71. The method of any one of the enumerated embodiments, wherein the purging formulation has a viscosity of about 0.5-0.6, about 0.6-0.7, about 0.7-0.8, about 0.8-0.9, about 0.9-1, about 1-2, about 2-3, about 3-4, about 4-5, about 5-6, about 6-7, about 7-8, about 8-9, about 9-10, about 10-20, about 20-30, about 30-40, about 40-50, about 50-60, about 60-70, about 70-80, about 80-90, about 90-100, about 100-120, about 120-140, about 140-160, about 160-180, about 180-200, about 200-250, about 250-300, about 300-350, about 350-400, about 400-450, about 450-500, about 550-600, about 600-650, about 650-700, about 700-750, about 750-800, about 800-850, about 850-900, about 900-950, or about 950-1000 centipoise (cP).
Enumerated Embodiment 72. The method of any one of the enumerated embodiments, wherein the purging formulation forms a bolus within the olfactory cleft.
Enumerated Embodiment 73. The method of any one of the enumerated embodiments, wherein the purging formulation forms a capillary bridge at a base of the olfactory cleft.
Enumerated Embodiment 74. The method of any one of the enumerated embodiments, wherein the purging formulation dilutes a bolus already within the olfactory cleft.
Enumerated Embodiment 75. The method of any one of the enumerated embodiments, wherein the purging formulation physically displaces a substance from the olfactory cleft.
Enumerated Embodiment 76. The method of any one of the enumerated embodiments, wherein the substance comprises a therapeutic agent.
Enumerated Embodiment 77. The method of any one of the enumerated embodiments, wherein the purging formulation purges at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of a substance from the olfactory cleft.
Enumerated Embodiment 78. The method of any one of the enumerated embodiments, wherein the purging formulation impedes transfer of an intranasal therapeutic agent to the olfactory cleft by reducing or eliminating contact between the therapeutic agent and a tissue within the olfactory cleft. The method of any one of the proceeding claims, wherein the purging formulation reduces a rate of drug transmission via the olfactory cleft.
Enumerated Embodiment 79. The method of any one of the enumerated embodiments, wherein the purging formulation exerts a pressure on a substance within the olfactory cleft to purge the substance from the olfactory cleft.
Enumerated Embodiment 80. The method of any one of the enumerated embodiments, wherein the purging formulation draws a substance from within the olfactory cleft into the purging formulation.
Enumerated Embodiment 81. The method of any one of the enumerated embodiments, wherein the purging formulation has a sufficiently low osmolality to draw a substance from within the olfactory cleft into the purging formulation.
Enumerated Embodiment 82. The method of any one of the enumerated embodiments, wherein the purging formulation is configured to cause an osmotic pressure sufficient to reverse a direction of a drug flow through a tissue of the olfactory cleft.
Enumerated Embodiment 83. The method of any one of the enumerated embodiments, wherein the purging formulation comprises water.
Enumerated Embodiment 84. The method of any one of the enumerated embodiments, wherein the purging formulation comprises one or more surfactants.
Enumerated Embodiment 85. The method of any one of the enumerated embodiments, wherein the purging formulation comprises one or more flavoring agents.
Enumerated Embodiment 86. The method of any one of the enumerated embodiments, wherein the purging formulation is configured to neutralize a therapeutic agent.
Enumerated Embodiment 87. The method of any one of the enumerated embodiments, wherein the purging formulation has a pH sufficient to cause degradation of a therapeutic agent.
Enumerated Embodiment 88. The method of any one of the enumerated embodiments, wherein the purging formulation comprises an enzyme configured to cause degradation of a therapeutic agent.
Enumerated Embodiment 89. The method of any one of the enumerated embodiments, wherein the purging formulation comprises a therapeutic agent configured for absorption into a tissue in the respiratory nasal cavity or the gastrointestinal tract.
Enumerated Embodiment 90. The method of any one of the enumerated embodiments, wherein the purging formulation is sterilized prior to delivery to the olfactory cleft.
Enumerated Embodiment 91. The method of any one of the enumerated embodiments, wherein the purging formulation is delivered as a single volume.
Enumerated Embodiment 92. The method of any one of the enumerated embodiments, wherein the purging formulation is delivered as multiple separate volumes.
Enumerated Embodiment 92. The method of any one of the enumerated embodiments, wherein a first volume of purging formulation is delivered to the olfactory cleft and a second volume of purging formulation is delivered to the olfactory cleft after waiting for a duration of time. Enumerated Embodiment 93. The method of any one of the enumerated embodiments, wherein the purging formulation is delivered via a designated left or right applicator configured for delivery to a left or right olfactory cleft.
Enumerated Embodiment 94. The method of any one of the enumerated embodiments, wherein the purging formulation is delivered selectively to a left or a right olfactory cleft.
Enumerated Embodiment 95. The method of any one of the enumerated embodiments, wherein the purging formulation is delivered selectively to a left or a right olfactory cleft and a second purging formulation is delivered selectively to the other olfactory cleft.
Enumerated Embodiment 96. A method of purging left and right olfactory clefts of a patient, the method comprising:
   a. providing a left purging formulation configured to purge the left olfactory cleft;
   b. providing a right purging formulation configured to purge the right olfactory cleft;
   c. delivering the left purging formulation to the left olfactory cleft, thereby purging the left olfactory cleft; and
   d. delivering the right purging formulation to the right olfactory cleft, thereby purging the right olfactory cleft.
Enumerated Embodiment 97. The method of any one of the enumerated embodiments, wherein the left purging formulation and the right purging formulation are identical.
Enumerated Embodiment 98. The method of any one of the enumerated embodiments, wherein the left purging formulation and the right purging formulation are different.
Enumerated Embodiment 99. The method of any one of the enumerated embodiments, wherein the left purging formulation and the right purging formulation are delivered at the same time.
Enumerated Embodiment 100. The method of any one of the enumerated embodiments, wherein the left purging formulation and the right purging formulation are delivered at different times.
Enumerated Embodiment 101. The method of any one of the enumerated embodiments, wherein the left purging formulation and the right purging formulation are delivered in equal volumes.
Enumerated Embodiment 102. The method of any one of the enumerated embodiments, wherein the left purging formulation and the right purging formulation are delivered in different volumes.
Enumerated Embodiment 103. A method of controlling delivery of a therapeutic formulation via an olfactory cleft of a patient, the method comprising:
   a. delivering a first volume of a therapeutic formulation to the olfactory cleft;
   b. monitoring a response to the therapeutic formulation; and
   c. delivering one or more additional volumes of the therapeutic formulation to the olfactory cleft based on the monitored response to the therapeutic formulation.
Enumerated Embodiment 104. The method of any one of the enumerated embodiments, wherein the response is monitored via a body temperature, a blood pressure measurement, a heart rate measurement, a respiration rate measurement, a blood oxygenation level, electroencephalography (EEG), or observation of pupil dilation.
Enumerated Embodiment 105. The method of any one of the enumerated embodiments, wherein the purging formulation is delivered via a device comprising:
   a. a housing defining first and second insertable portions, each for insertion into a nasal channel of the subject, wherein, upon insertion of the first insertable portion into the nasal channel of the subject, the at least one insertable portion engages tissue within the nasal channel to open or expand an internal nasal valve of the subject thereby positioning at least one of the insertable portions for delivery of the purging formulation to the olfactory region of the subject; and
   b. an actuator which delivers the purging formulation from the either or both of the insertable portions when the device is actuated.
Enumerated Embodiment 106. A device for purging an olfactory cleft of a patient, the device comprising:
   a. a housing comprising an insertable portion comprising a distal end, and a proximal end; and
   b. a subject-engaging portion which engages a columella region of the subject to seat the distal end of the insertable portion within an ejection zone of a nasal channel of the subject;
wherein the device is configured to deliver a purging formulation to the olfactory cleft of the patient, thereby purging the olfactory cleft.
Enumerated Embodiment 107. A device for controlled delivery of a therapeutic formulation via an olfactory cleft of a patient, the device comprising:
   a. a first chamber configured to hold the therapeutic formulation;
   b. a second chamber configured to hold a purging formulation for purging the therapeutic formulation from the olfactory cleft; and
   c. a delivery mechanism configured to sequentially deliver the therapeutic formulation and the purging formulation to the olfactory cleft.
Enumerated Embodiment 108. The device of any one of the enumerated embodiments, additionally comprising a dispensing tip configured to allow for precision delivery of the therapeutic formulation and the purging formulation to the olfactory cleft.
Enumerated Embodiment 109. The device of any one of the enumerated embodiments, wherein the device is configured to eject each of the therapeutic formulation and the purging formulation as sequential laminar jets.
Enumerated Embodiment 110. The method of any one of the enumerated embodiments, wherein the purging formulation is ejected from a delivery device as a flow having a Reynold's number of 2300 or less.
Enumerated Embodiment 111. The method of any one of the enumerated embodiments, wherein the duration of time can be selected based on monitoring of a response to the therapeutic formulation. Enumerated Embodiment 112. The method of any one of the enumerated embodiments, wherein the duration of time can be at least about 15 seconds, at least about 30 seconds, at least about 45 seconds, at least about 60 seconds, at least about 90 seconds, at least about 120 seconds, at least about 150 seconds, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 40 minutes, at least about 50 minutes, at least about 60 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, or longer than 24 hours.
Enumerated Embodiment 113. The method of any one of the enumerated embodiments, wherein a volume of the purging formulation delivered to the olfactory cleft is less than about 5000 uL, less than about 4500 uL, less than about 4000 uL, less than about 3500 uL, less than about 3000 uL, less than about 2500 uL, less than about 2000 uL, less than about 1500 uL, less than about 1000 uL, less than about 900 uL, less than about 800 uL, less than about 700 uL, less than about 600 uL, less than about 500 uL, less than about 400 uL, less than about 300 uL, less than about 200 uL, less than about 100 uL, less than about 90 uL, less than about 80 uL, less than about 70 uL, less than about 60 uL, less than about 50 uL, less than about 40 uL, less than about 30 uL, less than about 20 uL, or less than about 10 uL.
Enumerated Embodiment 114. The method of any one of the enumerated embodiments, wherein the purging formulation purges at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the therapeutic formulation remaining at the olfactory cleft after the duration of time.
Enumerated Embodiment 115. A method of delivery of a therapeutic formulation via an olfactory cleft of a patient, the method comprising: a. delivering the therapeutic formulation to the olfactory cleft; and b. delivering a purging formulation to the olfactory cleft, thereby purging the therapeutic formulation from the olfactory cleft.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

The scope of the claims should not be limited by the embodiments and examples, but should be given the broadest interpretation consistent with the description as a whole.

1. A device for controlled delivery of a therapeutic formulation via an olfactory cleft of a subject, the device comprising:
a) a first chamber configured to hold the therapeutic formulation;
b) a second chamber configured to hold a purging formulation for purging the therapeutic formulation from the olfactory cleft; and
c) a delivery mechanism configured to sequentially deliver the therapeutic formulation and the purging formulation to the olfactory cleft.

2. The device of claim 1, additionally comprising a dispensing tip configured to allow for precision delivery of the therapeutic formulation and the purging formulation to the olfactory cleft.

3. The device of claim 1, wherein the device is configured to eject each of the therapeutic formulation and the purging formulation as sequential laminar jets.

4. The device of claim 1, further comprising a timer for measurement of a predetermined residence time between delivery of the therapeutic formulation and delivery of the purging formulation.

5. A method of delivering a therapeutic formulation via an olfactory cleft of a subject, the method comprising:
a) delivering the therapeutic formulation to the olfactory cleft;
b) waiting for a duration of time; and
c) delivering a purging formulation to target the olfactory cleft, thereby purging the therapeutic formulation from the olfactory cleft.

6. The method of claim 5, wherein the delivering the purging formulation comprises dispensing the purging formulation from a delivery device as a laminar flow.

7. The method of claim 5, wherein the delivering the purging formulation comprises dispensing the purging formulation from an ejection zone in a nasal cavity of the subject, wherein the ejection zone is:
a) 0 mm to 30 mm superior to a horizontal line that intersects an anterior aspect of an internal nasal valve, and
b) 0 mm to 20 mm anterior to an inclined line that intersects an anterior aspect of a middle turbinate and a posterior aspect of a vestibule.

8. The method of claim 5, wherein the purging formulation and the therapeutic formulation are delivered via ejection from a delivery device, and wherein a volume of the purging formulation ejected is not more than 2-fold a volume of the therapeutic formulation.

9. The method of claim 5, wherein the purging formulation is delivered at a velocity of between about 3 m/s and about 15 m/s.

10. The method of claim 5, wherein the purging formulation forms a bolus within the olfactory cleft.

11. The method of claim 10, wherein the purging formulation is configured to exert a pressure on a substance within the olfactory cleft to purge the substance from the olfactory cleft.

12. The method of claim 10, wherein the purging formulation is configured to draw a substance from within the olfactory cleft into the purging formulation.

13. The method of claim 10, wherein the purging formulation is configured to cause an osmotic pressure sufficient to reverse a direction of a drug flow through a tissue of the olfactory cleft.

14. The method of claim 5, wherein the olfactory cleft is a left olfactory cleft and/or a right olfactory cleft, and wherein the purging formulation is delivered selectively to the left olfactory cleft or the right olfactory clef.

15. The method of claim 5, wherein the olfactory cleft is a left olfactory cleft and/or a right olfactory cleft, and wherein: (i) the purging formulation is delivered selectively to the left olfactory cleft and a second purging formulation is delivered selectively to the right olfactory cleft; and/or (ii) the purging formulation is delivered selectively to the right olfactory cleft and a second purging formulation is delivered selectively to the left olfactory cleft.

16. The method of claim 5, wherein the therapeutic formulation and the purging formulation are delivered via a single dual-chamber device.

17. The method of claim 5, wherein the therapeutic formulation and the purging formulation are delivered via a device configured to sequentially deliver the therapeutic formulation from a first primary drug container and the purging formulation from a secondary drug container.

18. The method of claim 5, wherein the duration time between the delivery of the therapeutic formulation and the delivery of the purging formulation is selected based on a monitored biomarker.

19. The method of claim 5, wherein the duration time between the delivery of the therapeutic formulation and the delivery of the purging formulation is selected based on a monitored biomarker selected from a group consisting of: a body temperature, a blood pressure measurement, a heart rate measurement, a respiration rate measurement, a blood oxygenation level, electroencephalography (EEG), and observation of pupil dilation.

20. The method of claim 5, wherein the purging formulation is delivered via a device comprising:
a housing defining a first insertable portion and a second insertable portion, each of the first insertable portion and the second insertable portion for insertion into a nasal channel of the subject, wherein, upon the insertion of the first insertable portion into the nasal channel of the subject, at least one of the first insertable portion and the second insertable portion engages tissue within the nasal channel to open or expand an internal nasal valve of the subject thereby positioning at least one of the first insertable portion and the second insertable portion for delivery of the purging formulation to an olfactory region of the subject; and
an actuator which delivers the purging formulation from either or both of the first insertable portion and the second insertable portions when the device is actuated.
